(19) United States
(12) Patent Application Publication
(10) Pub. No.: US 2022/0000873 A1
(43) Pub. Date: Jan. 6, 2022

(54) S3QELS TO PROTECT AGAINST INTESTINAL PERMEABILITY

(71) Applicant: Buck Institute for Research on Aging, Novato, CA (US)

(72) Inventors: Mark A. Watson, Northfield, Birmingham (GB); Martin D. Brand, San Rafael, CA (US); Pankaj Kapahi, Kensington, CA (US)

(73) Assignee: Buck Institute for Research on Aging, Novato, CA (US)

(21) Appl. No.: 17/311,975

(22) PCT Filed: Dec. 9, 2019
(86) PCT No.: PCT/US2019/065292

§ 371 Date: Jun. 8, 2021

Related U.S. Application Data
(60) Provisional application No. 62/778,868, filed on Dec. 12, 2018.

(51) Int. Cl. A61K 31/519; A61K 31/517; A61K 31/343; A61K 31/4365; A61K 31/495; A61K 31/445; A61P 1/00; A61P 3/04

(52) U.S. Cl. CPC A61K 31/519; A61K 31/517; A61K 31/343; A61P 3/04; A61K 31/495; A61K 31/445; A61P 1/00; A61K 31/4365

(57) ABSTRACT

In various embodiments methods for the treatment or prophylaxis of an age-related and/or pathology-associated increase in intestinal barrier permeability are provided. In certain embodiments the methods comprise administering to a mammal in need thereof an effective amount of one or more agent(s) that inhibit superoxide or hydrogen peroxide production from the outer ubiquinone-binding site of complex III of the mitochondrial electron transport chain (site III_{Qo}).

### CROSS-REFERENCE TO RELATED APPLICATIONS

This application claims benefit of and priority to U.S. Ser. No. 62/778,868, filed on Dec. 12, 2018, which is incorporated herein by reference in its entirety for all purposes.

### STATEMENT OF GOVERNMENTAL SUPPORT

This invention was made with government support under Grant No. R01 AG045835 and R56 AG038688 awarded by the National Institutes of Health. The Government has certain rights in this invention.

### INCORPORATION BY REFERENCE OF SEQUENCE LISTING PROVIDED AS A TEXT FILE

A Sequence Listing is provided herewith as a text file, “BUCK-P066PUS_ST25.TXT” created on Nov. 8, 2019 and having a size of 8.13 kb. The contents of the text file are incorporated by reference herein in their entirety.

### BACKGROUND

The human intestinal barrier covers a surface of about 400 m² and represents approximately 10% of the body's energy expenditure. The intestinal barrier prevents loss of water and electrolytes from the body and blocks entry of antigens and microorganisms into the body (Brandtzaeg (2011) Eur. J Pharmacol. 668(Suppl 1): S16-S32) while allowing exchange of molecules between the subject and environment and facilitating absorption of nutrients in the diet. Specialized adaptations of the mammalian intestinal mucosa fulfill two seemingly opposing functions: First the intestine appears to be adapted to permit a peaceful co-existence with intestinal symbionts without eliciting chronic inflammation. Second, the intestine provides a measured inflammatory and defensive response to various pathogens (see, e.g., Hooper et al. (2012) Science 336: 1268-1273; Maynard et al. (2012) Nature, 489: 231-241). The intestinal barrier is a complex multilayer system, consisting of an external “physical” barrier and an inner “functional” and “immunological” barrier. The interaction of these two barriers enables a functional and useful permeability to be maintained (Scaldaferri et al. (2012) J. Clin. Gastroenterol. 46(Suppl): S12-S17).

There is now increasing evidence that loss of intestinal barrier function can occur either abruptly, e.g. following a major trauma resulting, e.g., in gram-negative sepsis and multi-organ failure (MOF), or gradually leading to chronic inflammatory, and other, diseases.

Although an apparent link exists between the function of the intestinal barrier and various diseases, the mechanisms are not well understood. For example, we have limited knowledge of what initially causes intestinal barrier dysfunction, and what prevents or restores it. The former might involve different events including, but not limited to, virus infections, reduced perfusion of the mucosa, drugs, or changes in the microbiota (see, e.g., Bischoff (2011) BMC Med. 9: 24). Recent data suggest that intestinal barrier and intestinal microbiota play a role in many different diseases such as idiopathic liver fibrosis or intestinal dysbiosis, the mechanisms of which have been unclear until recently (see, e.g., Tremellen et al. (2012) Med. Hypotheses, 79: 104-112; Seki & Schnabl (2012) J. Physiol. 590: 447-458; Serino et al. (2012) Gut, 61: 543-553; and the like).

### SUMMARY

The underlying causes of aging remain elusive, but may include decreased intestinal homeostasis followed by disruption of the intestinal barrier, which can be mimicked by nutrient-rich diets. S3QELs are small-molecule suppressors of site III_{Qo} electron leak. They suppress superoxide (or hydrogen peroxide) generation at complex III of the mitochondrial electron transport chain without inhibiting oxidative phosphorylation. As described herein, we show that feeding different S3QELs to Drosophila on a high-nutrient diet protects against greater intestinal permeability and apoptotic cell number, and against shorter median lifespan. Feeding S3QELs to mice on a high-fat diet also protects against the diet-induced increase in intestinal permeability. Our results demonstrate that superoxide superoxide (or hydrogen peroxide) produced by complex III in enterocytes is necessary and sufficient to cause diet-induced intestinal barrier disruption in both flies and mice.

Additionally the results described herein indicate that suppressors of site III_{Qo} electron leak can be administered prophylactically to delay or to prevent an onset of increase in intestinal permeability, or therapeutically to reduce or eliminate intestinal permeability, or to slow or stop an increase in intestinal permeability.

Accordingly, various embodiments contemplated herein may include, but need not be limited to, one or more of the following:

Embodiment 1: A method for the treatment or prophylaxis of an age-related and/or pathology-associated increase in intestinal barrier permeability, said method comprising:
administering to a mammal in need thereof an effective amount of one or more agent(s) that inhibit superoxide production from the outer ubiquinone-binding site of complex III of the mitochondrial electron transport chain (site III_{Qo}).

Embodiment 2: The method of embodiment 1, wherein said agent comprises an agent that partially or fully suppresses superoxide generation at complex III of the mitochondrial electron transport chain without inhibiting oxidative phosphorylation.

Embodiment 3: The method of embodiment 2, wherein said agent comprises a small-molecule suppressor of site III_{Qo} electron leak (a S3QEL).

Embodiment 4: The method according to any one of embodiments 1-3, wherein said agent comprises a sulfanyloxoquinazoline structural group S3QEL.

Embodiment 5: The method of embodiment 4, wherein said agent comprises a S3QEL selected from the group consisting of S3QEL-1, S3QEL-1.1, S3QEL-1.2, and S3QEL-1.3.

Embodiment 6: The method according to any one of embodiments 1-5, wherein said agent comprises a pyrazolopyrimidine structural group S3QEL.

Embodiment 7: The method of embodiment 6, wherein said agent comprises a S3QEL selected from the group consisting of S3QEL-2, S3QEL-2.1, S3QEL-2.2, S3QEL-2.3, S3QEL-2.4, S3QEL-2.5, S3QEL-2.6, S3QEL-2.7, S3QEL-2.8.

Embodiment 8: The method according to any of embodiments 1-7, wherein said agent comprises a S3QEL selected from the group consisting of S3QEL-4, S3QEL-5, S3QEL-6, and S3QEL-7.

Embodiment 9: The method according to any one of embodiments 1-3, wherein said agent comprises S3QEL-1, S3QEL-1.1, S3QEL-1.2, and S3QEL-1.3.

Embodiment 10: The method of embodiment 9, wherein said agent comprises S3QEL-2.

Embodiment 11: The method according to any one of embodiments 9-10, wherein said agent comprises S3QEL-2.1.

Embodiment 12: The method according to any one of embodiments 9-11, wherein said agent comprises S3QEL-2.2.

Embodiment 13: The method according to any one of embodiments 9-12, wherein said agent comprises, S3QEL-2.3.

Embodiment 14: The method according to any one of embodiments 9-13, wherein said agent comprises S3QEL-2.4.

Embodiment 15: The method according to any one of embodiments 9-14, wherein said agent comprises S3QEL-2.5.

Embodiment 16: The method according to any one of embodiments 9-15, wherein said agent comprises S3QEL-2.6.

Embodiment 17: The method according to any one of embodiments 9-16, wherein said agent comprises S3QEL-2.7.

Embodiment 18: The method according to any one of embodiments 9-17, wherein said agent comprises S3QEL-2.8.

Embodiment 19: The method according to any one of embodiments 9-18, wherein said agent comprises S3QEL-4.

Embodiment 20: The method according to any one of embodiments 9-19, wherein said agent comprises S3QEL-5.

Embodiment 21: The method according to any one of embodiments 9-20, wherein said agent comprises S3QEL-6.

Embodiment 22: The method according to any one of embodiments 9-21, wherein said agent comprises S3QEL-7.

Embodiment 23: The method according to any one of embodiments 1-22, wherein said agent(s) are provided in a delivery vehicle compatible with a hydrophobic compound, a pharmaceutically acceptable solvate, a pharmaceutically acceptable ester or ether, or a pharmaceutically acceptable clathrate.

Embodiment 24: The method of embodiment 23, wherein said agent(s) are provided as a lipid or liposome formulation.

Embodiment 25: The method according to any one of embodiments 1-24, wherein said method provides treatment or prophylaxis for diet-induced intestinal permeability.

Embodiment 26: The method of embodiment 25, wherein said method provides treatment for diet-induced intestinal permeability in mammal that is obese or clinically obese.

Embodiment 27: The method of embodiment 25, wherein said method provides treatment for diet-induced intestinal permeability in a mammal that is diabetic.

Embodiment 28: The method of embodiment 25, wherein said method provides prophylaxis for diet-induced intestinal permeability in a mammal that is obese or clinically obese.

Embodiment 29: The method of embodiment 25, wherein said method provides prophylaxis for diet-induced intestinal permeability in mammal that is diabetic.

Embodiment 30: The method according to any one of embodiments 1-24, wherein said method provides treatment for age-related increases in intestinal permeability.

Embodiment 31: The method according to any one of embodiments 1-24, wherein said method provides treatment or prophylaxis for a pathology-associated increase in intestinal barrier permeability.

Embodiment 32: The method of embodiment 31, wherein said pathology comprises a pathology directly associated with the gut.

Embodiment 33: The method of embodiment 32, wherein said pathology comprise a pathology selected from the group consisting of a gastric ulcer, infectious diarrhea, irritable bowel syndrome (IBS), inflammatory bowel disease (IBD), celiac disease, cancer associated with digestive tract (esophagus, stomach, colorectal), colitis, Crohn's disease, mitochondrial neurogastrointestinal encephalopathy (MNGIE), and hyperintestinal permeability.

Embodiment 34: The method of embodiment 33, wherein said mammal is a mammal identified as having a gastric ulcer.

Embodiment 35: The method of embodiment 33, wherein said mammal is a mammal identified as having infectious diarrhea.

Embodiment 36: The method of embodiment 33, wherein said mammal is a mammal identified as having irritable bowel syndrome (IBS).

Embodiment 37: The method of embodiment 33, wherein said mammal is a mammal identified as having inflammatory bowel disease (IBD).

Embodiment 38: The method of embodiment 33, wherein said mammal is a mammal identified as having celiac disease.

Embodiment 39: The method of embodiment 33, wherein said mammal is a mammal identified as having cancer associated with digestive tract (e.g., esophagus, stomach, colorectal).

Embodiment 40: The method of embodiment 33, wherein said mammal is a mammal identified as having colitis.

Embodiment 41: The method of embodiment 33, wherein said mammal is a mammal identified as having Crohn's disease.

Embodiment 42: The method of embodiment 33, wherein said mammal is a mammal identified as having mitochondrial neurogastrointestinal encephalopathy (MNGIE).

Embodiment 43: The method of embodiment 33, wherein said mammal is a mammal identified as having hyperintestinal permeability.

Embodiment 44: The method of embodiment 31, wherein said pathology comprises a pathology indirectly associated with the gut.

Embodiment 45: The method of embodiment 44, wherein said pathology comprise a pathology selected from the group consisting of a respiratory infection, acute inflammation (sepsis, SIRS, MOF), chronic inflammation, and an obesity-associated metabolic disease (e.g., NASH, diabetes type I and II, CVD).

Embodiment 46: The method of embodiment 45, wherein said mammal is a mammal identified as having a respiratory infection.

Embodiment 47: The method of embodiment 45, wherein said mammal is a mammal identified as having an acute inflammation (sepsis, SIRS, MOF).

Embodiment 48: The method of embodiment 45, wherein said mammal is a mammal identified as having chronic inflammation.

Embodiment 49: The method of embodiment 45, wherein said mammal is a mammal identified as having an obesity-associated metabolic disease.

Embodiment 50: The method of embodiment 49, wherein said obesity-associated metabolic disease comprises one or more pathologies selected form the group consisting of (NASH, type I diabetes, type II diabetes, and CVD.

Embodiment 51: The method according to any one of embodiments 33-50, wherein said method ameliorates one or more symptoms of said pathology.

Embodiment 52: The method according to any one of embodiments 1-51, wherein said method is a therapeutic method to reduce intestinal permeability, or to slow or stop an increase in intestinal permeability.

Embodiment 53: The method according to any one of embodiments 1-51, wherein said method is a prophylactic method effective to delay or to prevent an onset of increase in intestinal permeability.

Embodiment 54: The method according to any one of embodiments 1-53, wherein said agent(s) do not decrease food consumption by said mammal.

Embodiment 55: The method according to any one of embodiments 1-54, wherein said agent(s) decrease the number of apoptotic cells in the intestine.

Embodiment 56: The method according to any one of embodiments 1-55, wherein said agent(s) decrease intestinal damage.

Embodiment 57: The method according to any one of embodiments 1-56, wherein said agent(s) decrease intestinal permeability.

Embodiment 58: The method of embodiment 57, wherein said decrease in intestinal permeability comprises a decrease in intestinal permeability as measured by presence of non-absorbable markers in serum after ingestion.

Embodiment 59: The method of embodiment 58, wherein said nonabsorbable markers are selected form the group consisting of nonabsorbable sugars, dextran, and PEGs.

Embodiment 60: The method of embodiment 59, wherein said nonabsorbable markers are selected from the group consisting of lactose, mannitol, L-rhamnose, sucralose, and erythritol.

Embodiment 61: The method of embodiment 59, wherein said nonabsorbable markers comprise FITC-dextran.

Embodiment 62: The method according to any one of embodiments 57-60, wherein said decrease in intestinal permeability comprises a decrease in intestinal permeability as measured by the presence or level of permeability biomarkers.

Embodiment 63: The method of embodiment 62, wherein said biomarkers comprise a biomarker selected from the group consisting of plasma zonulin, calprotectin, and alpha-1 antitrypsin (A1AT).

Embodiment 64: The method according to any one of embodiments 57-63, wherein said decrease in intestinal permeability comprises a decrease in intestinal permeability as measured by plasma lipopolysaccharide load.

Embodiment 65: The method according to any one of embodiments 57-64, wherein said decrease in intestinal permeability comprises a decrease in intestinal permeability as measured by circulating endotoxin core antibodies (EndoCAb) and/or plasma D-lactate level, and/or fecal butyrate concentration.

Embodiment 66: The method according to any one of embodiments 57-65, wherein said decrease in intestinal permeability comprises a decrease in intestinal permeability as measured by the presence or level of inflammatory cytokines.

Embodiment 67: The method of embodiment 66, wherein said inflammatory cytokines comprise a cytokine selected from the group consisting of TNFα, INFγ, IL-1β, and IL-13.

Embodiment 68: The method according to any one of embodiments 57-67, wherein said agent(s) decreases the expression of antimicrobial protein genes.

Embodiment 69: The method of embodiment 68, wherein said antimicrobial protein genes include one or more genes selected from the group consisting of Dpt, Drs and Def, and upd3.

Embodiment 70: The method according to any one of embodiments 1-69, wherein said agent(s) act in intestinal enterocytes.

Embodiment 71: The method of embodiment 70, wherein said agent(s) act specifically in intestinal enterocytes.

Embodiment 72: The method according to any one of embodiments 1-71, wherein administering said agent(s) decreases weight gain.

Embodiment 73: The method according to any one of embodiments 1-72, wherein said agent(s) are administered via a route selected from the group consisting of intraperitoneal administration, oral administration, inhalation administration, transdermal administration, subdermal depot administration, and rectal administration.

Embodiment 74: The method of embodiment 73, wherein said agent(s) are administered orally.

Embodiment 75: The method according to any one of embodiments 73-74, wherein said agent(s) are administered as a unit dosage formulation.

Embodiment 76: The method according to any one of embodiments 1-75, wherein said mammal is a human.

Embodiment 77: The method according to any one of embodiments 1-75, wherein said mammal is a non-human mammal.

Embodiment 78: A kit for the treatment or prophylaxis of an age-related and/or pathology-associated increase in intestinal barrier permeability, said kit comprising:
one or more agent(s) that inhibit superoxide production from the outer ubiquinone-binding site of complex III of the mitochondrial electron transport chain (site III_{Qo}); and
instructional materials describing the use of the active agent(s) in a method for the treatment or prophylaxis of an age-related and/or pathology-associated increase in intestinal barrier permeability.

Embodiment 79: The kit of embodiment 78, wherein said instructional materials teach the use of said agent(s) in a method according to any one of embodiments 1-77.

Embodiment 80: The kit according to any one of embodiments 78-79, wherein said agent(s) comprise one or more S3QELs selected from the group consisting of S3QEL-1, S3QEL-1.1, S3QEL-1.2, and S3QEL-1.3, S3QEL-2, S3QEL-2.1, S3QEL-2.2, S3QEL-2.3, S3QEL-2.4, S3QEL-2.5, S3QEL-2.6, S3QEL-2.7, S3QEL-2.8, S3QEL-4, S3QEL-5, S3QEL-6, and S3QEL-7.

### Definitions

The terms “mammal”, “subject,” “individual,” and “patient” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the subject can be a human (e.g., adult male, adult female, adolescent male, adolescent female, male child, female child) under the care of a physician or other health worker in a hospital, psychiatric care facility, as an outpatient, or other clinical context. In certain embodiments, the subject may not be under the care or prescription of a physician or other health worker.

As used herein, the phrase “a subject in need thereof” refers to a subject, as described infra, that suffers or is at a risk of suffering (e.g., pre-disposed such as genetically pre-disposed) from increased (above normal) intestinal permeability and/or the diseases or conditions associated with increased intestinal permeability.

An “effective amount” refers to an amount effective, at dosages and for periods of time necessary, to achieve the desired therapeutic or prophylactic result. A “therapeutically effective amount” may vary according to factors such as the disease state, age, sex, and weight of the individual, and the ability of the pharmaceutical to elicit a desired response in the individual. A therapeutically effective amount is also one in which any toxic or detrimental effects of a treatment are substantially absent or are outweighed by the therapeutically beneficial effects. In certain embodiments the term “therapeutically effective amount” refers to an amount of an active agent or composition comprising the same that is effective to “treat” a disease or disorder in a mammal (e.g., a patient or a non-human mammal). In one embodiment, a therapeutically effective amount is an amount sufficient to slow or stop, or prevent an increase in intestinal permeability and/or to improve at least one symptom associated with a pathology associated with increased intestinal permeability.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount is less than the therapeutically effective amount.

The terms “treatment,” “treating,” or “treat” as used herein, refer to actions that produce a desirable effect on the symptoms or pathology of a disease or condition, particularly those that can be effected utilizing the compositions described herein, and may include, but are not limited to, even minimal changes or improvements in one or more measurable markers of the disease or condition being treated. Treatments also refers to delaying the onset of, retarding or reversing the progress of, reducing the severity of, or alleviating or preventing either the disease or condition to which the term applies, or one or more symptoms of such disease or condition. “Treatment,” “treating,” or “treat” does not necessarily indicate complete eradication or cure of the disease or condition, or associated symptoms thereof. In one embodiment, treatment comprises improvement of at least one symptom of a disease being treated. The improvement may be partial or complete. The subject receiving this treatment is any subject in need thereof. Exemplary markers of clinical improvement will be apparent to persons skilled in the art.

The term “mitigating” or “ameliorating” refers to reduction or elimination of one or more symptoms of that pathology or disease, and/or a reduction in the rate or delay of onset or severity of one or more symptoms of that pathology or disease, and/or the prevention of that pathology or disease.

The “intestinal barrier” is a functional entity separating the gut lumen from the inner host, and consisting of mechanical elements (mucus, epithelial layer), humoral elements (defensins, IgA), immunological elements (lymphocytes, innate immune cells), muscular and neurological elements.

“Intestinal permeability” is defined as a functional feature of the intestinal barrier at given sites, measurable, inter alia, by analyzing flux rates across the intestinal wall as a whole or across wall components, e.g., as described herein.

“Normal intestinal permeability” refers to a stable permeability found in healthy individuals with no signs of intoxication, inflammation or impaired intestinal functions.

“Impaired (increased) intestinal permeability” refers to a disturbed permeability being non-transiently changed compared to the normal permeability leading to a loss of intestinal homeostasis, functional impairments and disease.

### BRIEF DESCRIPTION OF THE DRAWINGS

FIG. 1 shows illustrative, but non-limiting examples of S3QELs.

FIG. 2, panels A-K, shows the effects of diet on intestines, lifespan and gene expression in Drosophila. After eclosion Drosophila were raised for five days on standard yeast medium then switched on day 5 to diets containing 0.5-5% (w/v) yeast extract (YE). Effects of YE % on (panel A) intestinal permeability in w¹¹¹⁸ flies, (panel B) number of intestinal apoptotic cells, and (panel C) number of intestinal proliferating stem cells measured using phosphohistone H3 (PH3). (panels D-H) Relationships between median lifespan, intestinal permeability, intestinal apoptotic number and intestinal PH3-positive cell number of w¹¹¹⁸ and canton S flies. Lines were fit using either linear regression (D-G) or exponential growth (H). Panels I-K) Effect of YE % on intestinal gene expression normalized to Rp49 then expressed as fold change relative to 5% YE. Panels show (panel I) inflammatory and damage markers (panel J) intestinal tight-junction genes and (panel K) antioxidant genes. Data are means±SEM of n=3 biological replicates each using 200 flies (panel A), or 12 (panels B, C) or 15 (panels I-K) dissected intestines. Dotted lines in panels A-C denote data points used for statistical analysis. *P<0.05, ***P<0.0001 by one-way ANOVA with Tukey's post-test (panels A-C) or two-way ANOVA with Dunnett's multiple comparisons test.

FIG. 3, panels A-J, shows the effects of S3QELs on intestines, lifespan and gene expression in Drosophila on 5% YE. After eclosion w¹¹¹⁸ Drosophila were raised for five days on standard yeast medium then switched on day 5 to diets containing 5% YE with either S3QEL or DMSO vehicle. Effects of S3QELs on (panel A) intestinal permeability at day 40 (FIG. 7 shows the full longitudinal measure), (panel B) number of intestinal apoptotic cells at day 30, (panel C) median lifespan (see FIG. 9 for lifespan curves). Panels D-G) Relationships between median lifespan, intestinal permeability, and intestinal apoptotic number between S3QEL- and DMSO-treated flies. Lines in D-G were fit using linear regression; arrows indicate directional trends. Panels H-J) Effect of S3QELs on intestinal gene expression normalized to Rp49 then expressed as fold change relative to DMSO vehicle. Panels show (panel H) inflammatory and damage markers (panel J) intestinal tight-junction genes and (panel K) antioxidant genes. Data are means±SEM of n=3 biological replicates each using 200 flies (panels A, C), 12 intestines (panel B) or 15 intestines (panels H-J). *P<0.05, ***P<0.0001 by one-way ANOVA with Tukey's post-test (H-J).

FIG. 4, panels A-F, shows the effects of S3QELs in intestinal-specific knockdown of superoxide dismutase (Sod). Intestinal enterocyte-specific knockdown of cytosolic Sod1 or mitochondrial Sod2 was initiated 5 days after eclosion when flies at 18° C. were transferred to 5% YE with either S3QEL or DMSO vehicle at 29° C. Effect of S3QELs on intestinal permeability (panels A, C), number of intestinal apoptotic cells (panels B, D), and lifespan (panels E, F) in Sod1 (panels A, B, E) and Sod2 (panels C, D, F) intestinal enterocyte knockdown. Data are means±SEM of n=3 biological replicates each using 200 flies (panels A, C, E, F), or 12 intestines (panels B, D). *P<0.05, **P<0.005, ***P<0.0001 by one-way ANOVA with Dunnett's multiple comparisons test (panels A-F). Lifespan curves in (panels E, F) were analyzed using the log-rank (Mantel-Cox) test. Shaded boxes in (panels A, C, E, F) indicate the % change from DMSO-treated; arrows give numerical values. Shaded boxes in lifespan graphs (panels E-F) indicate the 5-day post-eclosion period before flies were transferred to 5% YE. AUC, area under the curve (% x days).

FIG. 5, panels A-F, shows the effects of S3QELs on C57BL/6 mice fed a high-fat diet. C57BL/6 mice were fed chow (Ctrl) or high-fat (HF) diet ±200 mg/kg S3QELs, for 16 weeks. Effects of S3QELs on (panel A) plasma FITC-Dextran after oral gavage and (panel B) albumin in fecal matter. Panels C, D, F) Effect of S3QELs on colonic expression of (panel C) tight-junction genes, (panel D) mucin genes, and (panel F) an ER stress gene, normalized to β-actin then expressed as fold change relative to control diet-fed mice. Panel E) Effect of S3QELs on glucose tolerance (individual time point glucose tolerance curves are shown in FIG. 12). Data are means±SEM of n=10 mice. Panels A, B) ***P<0.0001 by two-way ANOVA with Dunnett's multiple comparison, for AUC *P<0.05 by one-way ANOVA with Tukey's post test. Panels C, D, F) *P<0.05, **P<0.009, ***<0.0003 by one-way ANOVA with Tukey's post-test. Panel E) ***P<0.0001 by two-way ANOVA with Dunnett's multiple comparison. ns, not significant. AUC, area under the curve.

FIG. 6, panels A-G, shows the effects of diet on lifespan, intestines and gene expression in Drosophila. After eclosion Drosophila were raised for five days on standard yeast medium (shaded bars), then switched on day 5 to diets containing 0.5-5% YE. Effects of YE % on lifespan in (panel A) w¹¹¹⁸ and (panel B) Canton S flies and on (panel C) intestinal permeability, (panel D) intestinal apoptosis, and (panel E) intestinal stem cell proliferation in Canton S flies. Panels F, G) Day 30 intestinal gene expression on different YE % diets in w¹¹¹⁸ flies, normalized to Rp49 and expressed as fold change relative to 5% YE. Panels show (panel F) inflammatory and damage markers and (panel G) intestinal tight-junction genes. Data are means±SEM of n=3 biological replicates each using 200 flies (panels A, B), or 12 (panels C-E) or 15 (panels F-G) dissected intestines. Panels A, B) ***P<0.0001 by log-rank (Mantel-Cox) test for differences between lifespan curves. For median lifespan analysis ***P<0.0003, **P<0.005, *P<0.05, ns, not significant, by one-way ANOVA with Tukey's post-test. Shaded boxes in lifespan graphs (panels A, B) indicate the 5-day post eclosion period before flies were transferred to 5% YE. Dotted lines in (panels C-E) denote data points used for statistical analysis. Panels C-G) *P<0.05, ***P<0.0001 by one-way ANOVA with Tukey's post-test. AUC, area under the curve.

FIG. 7, shows the effect of S3QELs on intestinal permeability in w¹¹¹⁸ flies on 5% YE. After eclosion w¹¹¹⁸ Drosophila were raised for five days on standard yeast medium, then switched on day 0 to a diet containing 5% YE with S3QEL or DMSO. Panels show the effects of three structurally different S3QELs at a range of concentrations on intestinal permeability. Data are means±SEM of n=3 biological replicates each using 200 flies. ***P<0.0001 by two-way ANOVA with Dunnett's multiple comparison.

FIG. 8, panels A-F, shows the effects of S3QELs on intestinal apoptosis and proliferation on flies fed 5% YE. After eclosion w¹¹¹⁸ Drosophila were raised for five days on standard yeast medium, then switched on day 0 to diets containing 5% YE with S3QEL or DMSO. Effect of S3QELs on the number of intestinal apoptotic cells at (panel A) day 10, and (panel B) day 30. Panel C) Effect of S3QELs on the number of intestinal stem cells proliferating at day 30 as % of DMSO vehicle control. Panel D) Relationship between intestinal proliferation and intestinal permeability in S3QEL- and DMSO-treated flies. The line in D was fit using linear regression. Effects of S3QELs on the number of intestinal stem cells proliferating at (panel E) day 10, and (panel F) day 30. Data are means±SEM of n=3 biological replicates each using 12 intestines. ***P<0.0001 by one-way ANOVA with Tukey's post-test.

FIG. 9, shows the effect of S3QELs on lifespan in flies fed 5% YE. After eclosion w¹¹¹⁸ Drosophila were raised for five days on a standard yeast medium, then switched on day 5 to a diet containing 5% YE with S3QEL or DMSO. Panels show the effects of S3QELs at a range of different concentrations on lifespan. Shaded boxes in lifespan graphs indicate the 5-day post-eclosion period before flies were transferred to 5% YE and S3QEL treatment. Data are means±SEM of n=3 biological replicates each using 200 flies. *P<0.05, **P<0.003, ***P<0.0001 by log-rank (Mantel-Cox) test to analyze lifespan curves. *P<0.05, **P<0.003 by paired t-test to analyze median lifespan.

FIG. 10, shows the effect of S3QELs on food consumption of W¹¹¹⁸ flies fed 5% YE. Flies were raised on a 5% YE and either given DMSO as control or fed S3QELs at 8.0 μM. After 10 days of habituation on this food, food intake per fly was assayed by 15-min dye uptake. Data are means±SEM of n=10 samples (five from each of two biological replicates; each sample contained five homogenized flies). ns, not significant by one-way ANOVA with Tukey's post-test.

FIG. 11, panels A-C, shows the effect of S3QELs on gene expression in the small distal intestine in C57BL/6 mice fed a high-fat diet. C57BL/6 mice were fed chow (Ctrl) or high-fat (HF) diet ±200 mg/kg S3QELs, for 16 weeks. Effect of S3QELs on colonic expression of (panel A) tight-junction genes, (panel B) mucin genes, and (panel C) an ER stress gene, normalized to R-actin then expressed as fold change relative to control diet-fed mice. Data are means±SEM of n=10 mice. *P<0.05, **P<0.009, ***<0.0001 by one-way ANOVA with Tukey's post-test.

FIG. 12, panels A-D, shows the effect of S3QELs on metabolic measures in C57BL/6 mice fed a high-fat diet. C57BL/6 mice were fed chow (Ctrl) or high-fat (HF) diet ±200 mg/kg S3QELs for 16 weeks. Panel A) Effect of S3QELs on glucose tolerance measured by the ability of each mouse to clear an intraperitoneal injection of 2 mg/kg glucose. Effect of S3QELs on (panel B) weight, (panel C) body composition and (panel D) food consumption over a 16-week period. Data are means±SEM of n=10 mice. Panel A) ***P<0.0001, otherwise not significant, by two-way ANOVA with Dunnett's multiple comparison. Panel B) *P<0.05, **P<0.003, ***<0.0001, ns, not significant, by one-way ANOVA with Tukey's post-test.

### DETAILED DESCRIPTION

It is demonstrated herein that suppressors of site III_{Qo} electron leak (S3QELs) protect against greater intestinal permeability and apoptotic cell number, and against shorter median lifespan in Drosophila. Moreover, feeding S3QELs to mammals on a high-fat diet also protects against the diet-induced increase in intestinal permeability. The results described herein demonstrate that superoxide (or hydrogen peroxide) produced by complex III in enterocytes is necessary and sufficient to cause diet-induced intestinal barrier disruption in both flies and mice.

In view of these observations, in various embodiments one or more agent(s) that inhibit superoxide (or hydrogen peroxide) production from the outer ubiquinone-binding site of complex III of the mitochondrial electron transport chain (site III_{Qo}) are administered prophylactically to delay or to prevent an onset of increase in intestinal permeability, or are administered therapeutically to reduce or eliminate intestinal permeability, or to slow or stop an increase in intestinal permeability.

Thus, in an illustrative, but non-limiting embodiments, a method for the treatment or prophylaxis of an age-related and/or pathology-associated increase in intestinal barrier permeability is provided. In various embodiments the method involves administering to a mammal in need thereof an effective amount of one or more agent(s) that inhibit superoxide (or hydrogen peroxide) production from the outer ubiquinone-binding site of complex III of the mitochondrial electron transport chain (site III_{Qo}).

Thus, in certain embodiment the methods described herein provide treatment or prophylaxis for diet-induced intestinal permeability. In certain embodiments the methods described herein provide treatment for diet-induced intestinal permeability (e.g., in a subject that is obese or clinically obese). In certain embodiments the methods described herein provide treatment for diet-induced intestinal permeability in a subject that is diabetic. In certain embodiments the methods described herein provide prophylaxis for diet-induced intestinal permeability in a subject that is obese or clinically obese. In certain embodiments the methods described herein provide prophylaxis for diet-induced intestinal permeability in a subject that is diabetic. In certain embodiments the treatment slows or stops the increase in intestinal permeability, or reverses (decreases) intestinal permeability. In certain embodiments the treatment ameliorates one or more symptoms associated with obesity or complications thereof (e.g., diabetes, metabolic syndrome, NASH) etc.

In certain embodiments the methods described herein provide treatment for age-related increases in intestinal permeability.

In certain embodiments the methods described herein provide treatment or prophylaxis for a pathology-associated increase in intestinal barrier permeability. In certain embodiments the treatment slows or stops the increase in intestinal permeability, or reverses (decreases) intestinal permeability associated with the pathology. In certain embodiments the treatment ameliorates one or more symptoms associated with the pathology.

Pathologies Associated with Increase in Intestinal Barrier Permeability

In certain embodiments methods are provided for the treatment and/or prophylaxis of increased intestinal permeability associated with various pathologies. As indicated above, in various embodiments the methods involve administering an effective amount of one or more agent(s) that inhibit superoxide (or hydrogen peroxide) production from the outer ubiquinone-binding site of complex III of the mitochondrial electron transport chain (site III_{Qo}) (S3QELs). In certain embodiments the methods also provide therapeutic treatment that ameliorates one or more symptoms of a pathology associated with intestinal permeability.

Pathologies associated with increased intestinal permeability are well known to those of skill in the art. Such pathologies include, but are not limited to pathologies directly associated with the intestine and intestinal function, or extraintestinal pathologies that are not per se intestinal diseases (see, e.g., Table 1).

TABLE 1
Illustrative diseases associated with increased intestinal permeability.

| Intestinal | Extraintestinal |
|---|---|
| Gastric ulcers | |
| Colitis | |
| Crohn's disease | |
| Infectious diarrhea | Infections (e.g., respiratory) |
| Irritable bowel syndrome | Acute inflammation (sepsis, |
| | SIRS, MOF) |
| Functional GI diseases | |
| Inflammatory bowel disease, Celiac | Chronic inflammation (e.g. |
| disease | arthritis) |
| Cancer (esophagus, colorectal) | Obesity-associated metabolic |
| | diseases: Metabolic syndrome, |
| | NASH, diabetes type I and II, |
| | CVD |
| Mitochondrial neurogastrointestinal | CNS diseases (Parkinson's |
| encephalopathy (MNGIE disease) | disease, Alzheimer's disease) |
| Hyperintestinal permeability | Liver disease |

Among these, irritable bowel syndrome (IBS), inflammatory bowel IBD, obesity and metabolic diseases have experienced increasing attention. Additionally, other diseases such as celiac disease are classic examples of diseases that are related to intestinal permeability.

### Celiac Disease and Ulcerative Colitis (UC)

Intestinal barrier dysfunction is a main feature of celiac disease (CD) and ulcerative colitis (UC) (see, e.g., Brandtzaeg (2011) Eur. J. Pharmacol. 668(Suppl 1): S16-S32; Hering et al. (2012) J. Physiol. 590: 1035-1044; Mankertz et al. (2007) Curr. Opin. Gastroenterol. 23: 379-383). It has routinely been observed that that increased intestinal permeability precedes clinical manifestations of celiac disease, but is insufficient to cause disease suggesting other factors being involved. Leak flux diarrhea and a facilitated uptake of noxious antigens are two consequences resulting from an impaired epithelial barrier. Barrier perturbations in inflammatory bowel disease (IBD) comprise alterations in epithelial tight junctions, e.g. a reduced number of horizontal tight junction strands and an altered tight junction protein expression and subcellular distribution.

Additionally, prion protein, a ubiquitous cellular glycoprotein being involved in cell adhesion, was found to be dislocated in IBD supporting the concept that disrupted barrier function contributes to this disorder (see, e.g., Petit et al. (2012) Gastroenterology, 143: 122-132). Increased incidence of apoptotic events, epithelial cell shedding, as well as erosions and ulcerations can also add to that leakiness (see, e.g., Duckworth & Watson (2011) Meth. Mol. Biol. 763: 105-114).

These barrier defects have been attributed to enhanced activity of proinflammatory cytokines like TNFα, INFγ, IL-1β, and IL-13, which are frequently highly expressed in the chronically inflamed intestine.

Although the etiology of IBD is far from being clear, chronic inflammation is believed to result from an inadequate immune response as a consequence of genetic predisposition as well as changes in, and altered responses to the intestinal microbiota. On the other hand, an insufficient mucosal response to bacterial stimuli can result in an insufficient immune response towards intestinal pathogens.

Intestinal Permeability in Irritable Bowel Syndrome (IBS)

Intestinal barrier dysfunction has been found to play a pathogenic role not only in IBD, but also in IBS. There is evidence that increased intestinal permeability is related to low-grade inflammation, visceral hypersensitivity and pain in IBS (Camilleri et al. (2012) Am. J. Physiol. Gastrointest. Liver Physiol. 303: G775-G785). In diarrhea-predominant IBS (IBS-D), cytoskeleton condensation and enlarged intercellular spaces between epithelial cells have been observed providing the morphological basis for increased intestinal permeability in IBS. These structural changes have been observed to correlate both with mast cell activation and symptoms including diarrhea and pain severity (see, e.g., Martínez et al. (2013) Gut, 62: 1160-1168). These data confirm earlier observations derived from Ussing chamber experiments showing increased paracellular permeability in colon tissue of IBS patients (see, e.g., Piche et al. (2009) Gut, 58: 196-201).

Intestinal Permeability in Obesity and Fatty Liver Disease

A number of studies on the pathophysiology of obesity and associated metabolic diseases such as NAFLD and NASH, type 2 diabetes mellitus, and cardiovascular diseases, shows that such pathologies are related to the intestinal barrier function and the intestinal microbiota. In particular, it has been shown that metabolic diseases are linked to increased intestinal permeability and translocation of bacteria or bacterial products like endotoxin from the intestine to the liver and to other tissues (see, e.g., Lima et al. (2010) J. Pediatr. Gastroenterol. Nutr. 50: 309-315; Kerckhoffs et al. (2010) Dig. Dis. Sci. 55: 716-723; Burcelin et al. (2002) Am. J. Physiol. Endocrinol. Metab. 282: E834-E842; Lam et al. (2012) PLoS One, 7: e34233). It has become clear that the microbiota of obese (Ley et al. (2006) Nature, 444: 1022-1023; Turnbaugh et al. (2006) Nature, 444: 1027-1031) and diabetic (see, e.g., Qin et al. (2012) Nature, 490: 55-60) individuals differs from that of the healthy, lean population. In the meantime, evidence is growing suggesting that these alterations are of functional relevance.

Liver Disease

Liver disease is often times associated with increased intestinal permeability. A disruption of the gut barrier allows microbial products and viable bacteria to translocate from the intestinal lumen to extraintestinal organs. The majority of the venous blood from the intestinal tract is drained into the portal circulation, which is part of the dual hepatic blood supply. The liver is therefore the first organ in the body to encounter not only absorbed nutrients, but also gut-derived bacteria and pathogen associated molecular patterns (PAMPs). Chronic exposure to increased levels of PAMPs has been linked to disease progression during early stages and to infectious complications during late stages of liver disease (cirrhosis).

Intestinal Permeability in the Critically Ill Patient

Not only chronic diseases such as IBD, IBS and metabolic diseases, but also acute intestinal failure and gram-negative sepsis typically observed in the critically ill patient are associated with an impaired intestinal barrier and marked enhancement of intestinal permeability. For that reason, gram-negative sepsis and subsequent multiple organ failure (MOF) is a common cause of death in the intensive care unit (ICU) (see, e.g., Swank & Deitch (1996) World J. Surg. 20: 411-417). Such complications are seen in patients undergoing major abdominal surgery, but also in trauma patients, burn patients and other ICU patients (see, e.g., Derikx et al. (2008) PLoS One, 3: e3954; 238. Derikx et al. (2010) Crit. Care Med. 38: 133-137; de Haan et al. (2009) Crit. Care, 13: R86. Hypo-perfusion of the intestinal tract is regarded as the culprit of such complications. Therefore, such events occur also in patients suffering from acute CVD, acute intestinal ischemia of any cause, and acute enterocyte toxicity, e.g. in the course of chemotherapy (see, e.g., Derikx et al. (2006) J. Pediatr. Hematol. Oncol. 28: 267-269; Hanssen et al. (2008) Ann. Surg. 2248: 117-125). Also under physiological conditions, a hypo-perfusion of the gut can happen resulting in gut dysfunction, e.g. in the course of exercise (see, e.g., van Wijck et al. (2011) PLoS One, 6: e22366).

### Parkinson's Disease and Alzheimer's Disease.

Parkinson's disease (PD) is characterized by alpha-synucleinopathy that affects all levels of the brain-gut axis including the central, autonomic, and enteric nervous systems. Recently, it has been recognized that the brain-gut axis interactions are significantly modulated by the gut microbiota via immunological, neuroendocrine, and direct neural mechanisms. Dysregulation of the brain-gut-microbiota axis in PD may be associated with gastrointestinal manifestations frequently preceding motor symptoms, as well as with the pathogenesis of PD itself, supporting the hypothesis that the pathological process is spread from the gut to the brain. Excessive stimulation of the innate immune system resulting from gut dysbiosis and/or small intestinal bacterial overgrowth and increased intestinal permeability may induce systemic inflammation, while activation of enteric neurons and enteric glial cells may contribute to the initiation of alpha-synuclein misfolding. Additionally, the adaptive immune system may be disturbed by bacterial proteins cross-reacting with human antigens (see, e.g., Mulak & Bonaz (2015) World J. Gastroenterol. 21(37): 10609-10620).

More generally, changes in the gut microbiota have been linked to neurodegenerative diseases. The pathways are not clear, but appear to involve lipopolysaccharide (LPS) in the outer membrane of Gram-negative bacteria and other bacterial proinflammatory toxins. Increase in gut permeability cam enable LPS to enter the circulation and thence to cross the blood-brain barrier and enter the brain, where β-amyloid can trigger AD pathology. It has been observed that blood LPS levels are increased in patients with AD compared with healthy controls. Additionally, aging or disease increases gut permeability so toxins can enter the circulation, and it has been determined that they are in the brain within 15 minutes. Anerobic Gram-negative bacilli in the gut microbiota, such as Bacteroides fragilis and Escherichia coli, secrete the proinflammatory toxins amyloid, endotoxin, LPS, and sncRNA.

Additionally, microbiome-derived LPS is associated with specific downregulation of neurofilament light polypeptide leading to loss of arborisation of neurons in AD. Bacterial LPS can be detected in brain lysates from the hippocampus and superior temporal lobe neocortex of AD brains in higher amounts than in age-matched controls (see, e.g., Zhao et al. (2017) Front. Cell Infect. Microbiol. 7: 318). Human neuronal-glial cells incubated with LPS exhibit significantly decreased output of DNA transcription products.

Microbial metabolites, protein segregation and neuroinflammation can also contribute to the neurodegenerative pathway, and possible triggers might be an immune response mediated by LPS or microbial metabolites travelling along the vagus nerve. The term MAPRANOSIS—Microbiota Associated Proteopathy And Neuroinflammation was proposed to describe the process (Friedland & Chapman (2017) PLOS One doi.org/10.1371/journal.ppat.1006654).

The abundance of proinflammatory gut microbiota Escherichia/Shigella is increased in patients with cognitive impairment and brain amyloidosis when compared with β-amyloid-negative patients and controls. In contrast, a decrease in the abundance of the anti-inflammatory Eubacterium rectale is seen in patients with cognitive impairment and brain amyloidosis.

These findings are associated with higher blood levels of the pro-inflammatory cytokines interleukin (IL)-1β, IL-6, chemokine ligand 2 (CXCL2), and NLRP3; and a lower blood level of the anti-inflammatory cytokine IL-10 in patients with cognitive impairment and brain amyloidosis (Cattaneo et al. (2016) Neurobiol. Aging, 49: 60-6).

Decreased gut microbiota diversity and a distinct gut microbiota composition have also been reported in patients with AD compared with age- and sex-matched controls, with an increased abundance of Firmicutes and a decreased abundance of Bacteroidetes and Bifidobacterium.

### Active Agents.

In various embodiments the method described herein involve administration to a mammal (e.g., a human or a non-human mammal) in need thereof one or more agent(s) that inhibit superoxide (or hydrogen peroxide) production from the outer ubiquinone-binding site of complex III of the mitochondrial electron transport chain (site III_{Qo}). Mitochondria make ATP, but also leak electrons to produce superoxide and H₂O₂, reactive oxygen species (ROS) that have signaling functions and cause oxidative damage and pathology (see, e.g., Sena & Chandel (2012) Mol. Cell, 48: 158-167; Quinlan et al. (2013) Meth. Enzymol. 526: 189-217). The outer Q-binding site of respiratory complex III (site III_{Qo}) is implicated in the broadest range of ROS-mediated signaling and pathologies (see, e.g., Sena & Chandel (2012) supra.; Bleier & Dröse (2013) Biochim. Biophys. Acta 1827: 1320-1331) because its capacity is large and because it generates superoxide towards the cytosol, poising it to influence cellular events.

Extensive high-throughput chemical screening has been used to identify a number of compounds that are selective suppressors of site III_{Qo} electron leak (S3QELs). Typically, these agents suppress site III_{Qo} electron leak without otherwise altering energy metabolism. Multiple structural classes of S3QELs have been identified with similar effects on both superoxide (or hydrogen peroxide) production from complex III and downstream cellular signaling (see, e.g., Orr et al. (2015) Nat. Chem. Biol. 11(11): 834-836).

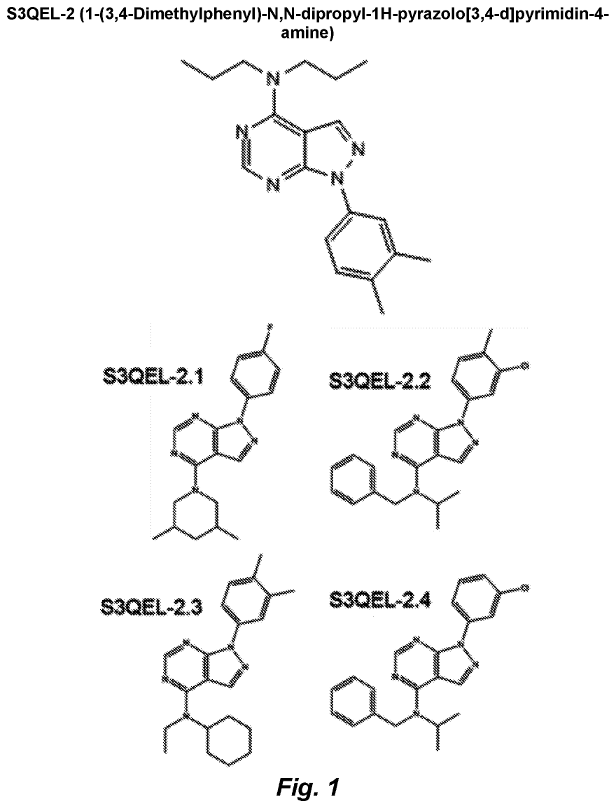

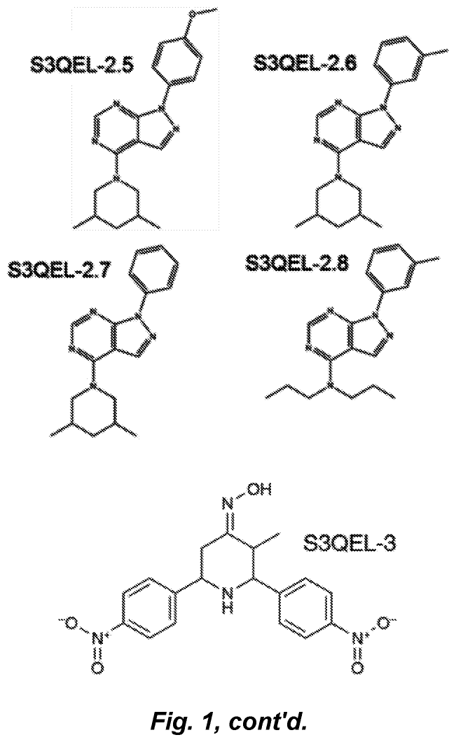

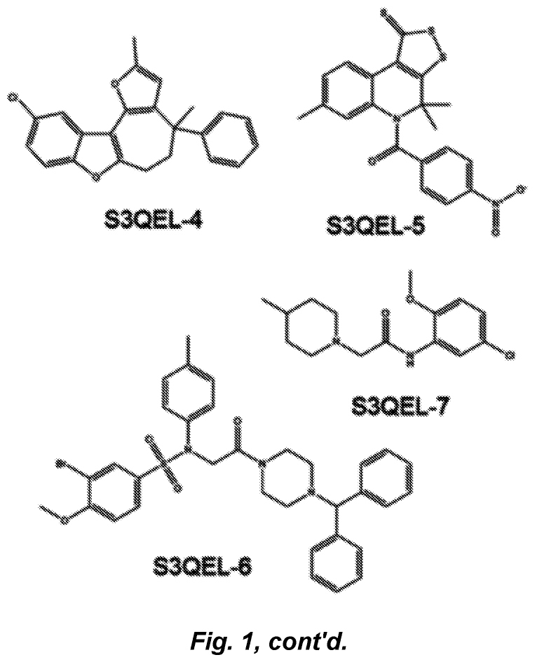

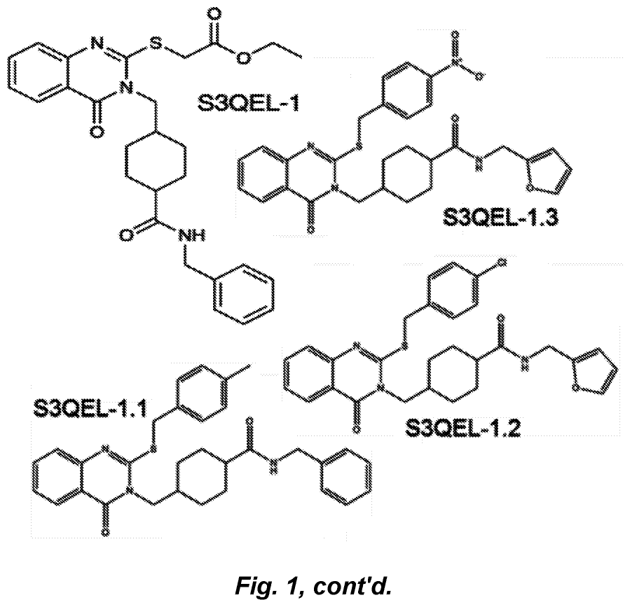

Illustrative S3QELs include, but are not limited to sulfanyloxoquinazoline structural group S3QELs (e.g., S3QEL-1 (e.g. S3QEL-1.1, S3QEL-1.2, S3QEL-1.3), pyrazolopyrimidine structural group S3QELs (e.g., S3QEL-2, S3QELs 2.1-2.8) and S3QELs 4-7 (see, e.g., FIG. 1).

S3QELs are commercially available from a number of suppliers. Thus, for example, the following S3QELs are available from Chemdiv: S3QEL-1 (catalog ID K284-4710), S3QEL-1.1 (K284-4711), S3QEL-1.2 (K284-4767), S3QEL-1.3 (K284-4794), S3QEL-2 (K405-3102), S3QEL-2.1 (Life Chemicals, F1886-0120), S3QEL-2.3 (K405-3096), S3QEL-2.4 (K405-3741), S3QEL-2.5 (K402-1025), S3QEL-2.6 (K402-0937), S3QEL-2.7 (K402-0893), S3QEL-2.8 (K402-0508), S3QEL-4 (3377-0061), S3QEL-5 (3389-0595), S3QEL-6 (3786-1206), S3QEL-7 (8010-6022). S3QEL-2.2 is available from Life Chemicals (Catalog No: F1886-0426), and S3QEL-3 is available from Maybridge (catalog No: JFD03367).

Additionally, S3QELS can readily be identified by screening protocols well known to those of skill in the art (see, e.g., Orr et al. (2015) Nat. Chem. Biol. 11(11): 834-836). As described therein, an Amplex UltraRed-based detection system was used to screen 635,000 small molecules against H₂O₂ production caused by electron leak at sites III_{Qo}, I_{Q} and II_{F} in isolated muscle mitochondria. Compounds that were unselective for site III_{Qo} or inhibited energy metabolism were eliminated. In various embodiments compounds are selected that selectively suppress site III_{Qo} superoxide (or hydrogen peroxide) production without impairing any tested measure of bioenergetic function, including mitochondrial membrane potential (Δψm). In the assays described by Orr et al. antimycin was used to induce strong superoxide (or hydrogen peroxide) production from site III_{Qo}. To determine whether S3QELs required antimycin for their action, they were tested against H₂O₂ production and three independent bioenergetic assays in mitochondria respiring on different substrates in the absence of antimycin. Desirable S3QELs suppress H₂O₂ production independently of either antimycin or respiratory substrate. The illustrative, but non-limiting screening strategy used by Orr et al. is summarized on Table 2.

TABLE 2
Illustrative, but non-limiting, screening strategy to identify S3QELs (see,
e.g., On et al. (2015) Nat. Chem. Biol. 11(11): 834-836).

| Step | Assay | Description | Criteria for selectivity |
|---|---|---|---|
| 1 | Primary | Endpoint H₂O₂ production | >40% inhibition of III_{Qo}; |
| | screen | from sites III_{Qo}, I_{Q}, II_{F} | <45% inhibition of II_{F}, |
| | | | <50% inhibition of I_{Q} |
| 2 | Confir- | Endpoint H₂O₂ production | >35% inhibition of III_{Qo}; |
| | mation | from sites III_{Qo}, I_{Q}, II_{F}. | <30% inhibition of I_{Q}, II_{F} |
| | screen | | |
| 3 | ΔΨm | Endpoint Δ_{Ψm} assay | <±30% effect |
| 4 | GalacTox | Endpoint cell viability | <20% decrease in cell |
| | | after 72 h exposure | viability |
| 5 | Dose- | Determine IC₅₀ against III_{Qo} | Progressive inhibition of |
| | response | superoxide production | III_{Qo}; (IC₅₀ < 3.2 μM) |
| | rescreen | | |
| 6 | Expanded | Determine selectivity in six | >30% suppression of |
| | rescreen | H₂O₂ and two Δ_{Ψm} endpoint | III_{Qo}; |
| | | assays | <30% inhibition in any |
| | | | other assay |
| 7 | Respiration | State 2, 3, 4o respiration | <30% inhibition of state |
| | | | 3; |
| | | | <30% increase in state 4o |
| 8 | Dose- | Confirm selectivity in six | Progressive, near complete |
| | response | H₂O₂ and two Δ_{Ψm} | suppression of III_{Qo}; |
| | with fresh | endpoint assays | <20% effect in any |
| | compounds | | other assay |

Using the assays described by Orr et al., or other similar assays, numerous S3QELS can readily be identified by one of skill in the art.

### Permeability Assays

As indicated above, in various embodiments one or more agent(s) that inhibit superoxide production from the outer ubiquinone-binding site of complex III of the mitochondrial electron transport chain (site III_{Qo}) are administered prophylactically to delay or to prevent an onset of increase in intestinal permeability, or therapeutically to reduce or eliminate intestinal permeability, or to slow or stop an increase in intestinal permeability. In certain embodiments this intestinal permeability comprises intestinal permeability assessed by one or more methods described below.

Administration of Non-Digestible Markers

In certain embodiments, this intestinal permeability can be assessed through enteral administration of non-digestible markers, which ideally should cross the mucosal barrier by non-mediated diffusion (Sun et al. (1998) Dig. Surg. 15: 386). The principle of this method is based on assessing the flow from the intestinal lumen to extraintestinal space such as blood, specific organs or urine. There are several types of markers including sugars, radioisotopes (e.g. ⁵¹Cr-EDTA) and polyethylene glycols (PEG).

In certain illustrative, but non-limiting embodiments, fluorescent-labeled dextrans can be used for assessment of intestinal permeability. Dextrans are polysaccharides and are available in different molecular sizes (e.g., ˜3 kD to ˜2000 kD) and conjugated to various fluorophores. Using a larger size will mimic bigger endogenous macromolecules, although dextran is still an inert test probe. It is important that tested tissue or blood does not have an autofluorescence that interferes with the emission of the fluorescent labeled probe. For example, during cholestatic liver disease, increased bilirubin in the plasma has a similar emission wavelength as fluorescein isothiocyanate-conjugated (FITC)-dextran. Choosing different fluorophores might overcome this problem. We are typically administering 200 μl of FITC-dextran 4 kD (600 mg/kg body weight) to mice by gavage, and the blood is collected 4 h later. Varying the time after harvesting will depend on which part of the intestinal tract is to be investigated. The serum concentration of the FITC-dextran is then determined using, e.g., a fluorimeter with an excitation wavelength of 490 nm and an emission wavelength of 530 nm. Serially diluted FITC-dextran can be used to establish a standard curve, and the concentration of serum FITC-dextran can then be calculated.

Non-metabolizable oligosaccharides have been introduced to develop reliable methods to assess the gut permeability (see, e.g., Menzies (1974) Biochem. Soc. Trans. Page 1042). The combined administration of a larger and a smaller molecule yields a specific large/small molecule ratio in the urine, which is a reflection of the intestinal permeability and has greater clinical value than the administration of one marker alone (Id.). The most common dual-sugar test in clinical practice is the lactulose-mannitol test (see, e.g., van Elburg et al. (1995) J. Pediatr. Gastroenterol. Nutr. 20: 184; Dastych et al. (2008) Dig. Dis. Sci. 53: 2789), however L-rhamnose is sometimes used instead of mannitol (see, e.g., van Nieuwenhoven et al. (1999) Eur. J. Clin. Investig. 29: 160-165; van Wijck et al. (2013) Clin. Nutr. 32: 245-251). Common characteristics of these sugars are that they are passively absorbed from the gut without considerable metabolism and that they are excreted in an unaltered form into the urine in a direct correlation to their absorbed amount from the intestine (see, e.g., Sequeira et al. (2014) PLoS One, 9: e99256). Mannitol is a monosaccharide with a molecular weight (MW) of ˜182 Da and demonstrates a transcellular permeation with high appearance in urine after oral application, and a decrease in the presence of villous atrophy as it occurs in celiac disease (see, e.g., Cobden et al. (1978) Br. Med. J. 2: 1060; Juby et al. (1989) Gut, 30: 476-480). Similarly, L-rhamnose, another monosaccharide of ˜164 Da, shows a reduced absorption in celiac disease, whereas lactulose is absorbed at an increased rate hence leading to a heightened lactulose/L-rhamnose excretion ratio (see, e.g., Menzies et al. (1979) Lancet, 2: 1107-1109). This is believed to occur since the larger disaccharide lactulose (MW 342 Da) is transported via a paracellular route through the gut wall versus the transcellular route of the aforementioned monosaccharides (see, e.g., Bjarnason et al. (1986) Dig. Dis. 4: 83-92; Maxton et al. (1986) Clin. Sci. 71: 71). In various embodiments, the lactulose-mannitol test involves the simultaneous ingestion of the sugars in water, and, after fasting for, e.g., 2 h, the collection of the urine over, e.g., a 24-hour period. The lactulose/mannitol ratio from the urine collection of the first 6 h can be used to measure the small intestinal permeability (see, e.g., Spiller et al. (2000) Gut, 47: 804-811). In certain embodiments urine collections at 0-3, 3-5, and 5-24 h can be carried out to assess the permeability of the proximal small intestine, distal small intestine, and colon, respectively.

In certain embodiments probes such as sucralose or erythritol, which remain unaffected by bacteria in the colon, are added to classical double sugar test, resulting in the so-called triple sugar test. The “multi sugar test” is based on administration of sucrose, lactulose, sucralose, erythritol, and rhamnose simultaneously in order to assess gastro-duodenal, small intestinal and large intestinal permeability in humans (see, e.g., van Wijck et al. (2013) Clin. Nutr. 32: 245-251).

Increased permeability for saccharides has been reported in patients with Crohn's disease, celiac disease, adverse reaction to food, and in critically ill patients or patients undergoing major surgery.

In contrast to lactulose and mannitol, PEGs have the advantage of being inert and can therefore be used to measure both small and large intestinal permeability. They have been used successfully to assess permeability changes in patients with irritable bowel syndrome, pancreatitis, liver cirrhosis, and intestinal ischemia reperfusion injury.

Laboratory analysis of urine samples is usually performed using high pressure liquid chromatography (HPLC) or liquid chromatography in combination with mass spectrometry (LC/MS).

Fecal Albumin

The use of assays that assess the flow from the blood to the intestinal lumen can also provide measures of intestinal permeability. Ideally endogenous markers are used that are restricted to the blood compartment during healthy conditions and are not present in the intestinal lumen. Following the onset of a barrier dysfunction this endogenous marker moves from blood vessels across the mucosal barrier into the intestinal lumen by non-mediated diffusion.

Measurement of albumin in fecal matter provides one illustrative example of such an assay. Albumin represents approximately 50% of the total protein content in human blood. Albumin is a small globular protein (molecular weight: 66.5 kDa) consisting of a single chain of 585 amino acids, produced by hepatocytes with no or very low intracellular storage (Nicholson et al., 2000; Evans, 2002). Approximately 30% to 40% of the albumin is maintained in the blood stream, while the remainder is distributed in the interstitial space, where its concentration is low (e.g., ˜1.4 g/dl). The protein leaves the circulation, returning to it via the lymphatic system.

Enhanced intestinal capillary permeability increases the release of albumin into the interstitial space. Typically for best accuracy, serum albumin levels need to be normal. Conditions with low serum levels of albumin due to decreased synthesis (e.g., end-stage liver disease) or due to albumin losing diseases (e.g., kidney disease) might result in false negative results. Usually an impermeable macromolecule in the healthy being, isotope-labeled albumin has been used to measure intestinal permeability in disease states. A healthy intestinal epithelial and endothelial barrier prevents the spilling over of albumin into the interstitial space. However, in conditions where the intestinal barrier integrity may be injured, endothelial and epithelial permeability is increased (Iqbal et al. (1996) Gut, 39: 199-203). Thereby, measurement of albumin in fecal samples is a good indicator of a disrupted intestinal barrier. In various illustrative, but non-limiting embodiments, albumin concentrations can be measured in freshly collected stool from mice by a standard ELISA test (Bethyl Lab).

Intestinal Biomarkers to Assess Intestinal Inflammation and Permeability

Any of a number of biomarkers can also be used to assess intestinal permeability. Illustrative biomarkers include, but are not limited to zonulin, calprotectin, Alpha-1-antitrypsin (A1AT), and the like.

Zonulin.

Zonulin is a 47 kDa protein that is believed to modulate intestinal permeability by disassembling intercellular tight junctions between epithelial cells in the digestive tract (see, e.g., Wang et al. (2000) J. Cell Sci. 113(Pt 24): 4435; Fasano (2001) Gut, 49: 159; Vanuytsel et al. (2013) Tissue barriers, 1: e27321). The effect of zonulin on increased intestinal permeability is mediated through activation of EGF receptor (EGFR) via proteinase-activated receptor 2 (PAR2) activation (Tripathi et al. (2009) Proc. Natl. Acad. Sci. USA, 106: 16799). Patients with type 1 diabetes, an autoimmune disease in which the finely tuned regulation of intestinal tight junctions is lost, have increased serum zonulin levels. Serum zonulin correlates with increased intestinal permeability. Moreover, zonulin upregulation appears to precede the onset of the disease, suggesting the important role of increased intestinal permeability in the pathogenesis of the disease (Sapone et al. (2006) Diabetes, 55: 1443). Other diseases with increased levels of zonulin are celiac disease and obesity (Fasano (2012), supra.).

Calprotectin.

Generally, defects or increased permeability of the mucosal barrier will cause intestinal inflammation in response to the enormous number of bacteria present in the bowel. Recruitment of leukocytes into the intestinal wall is important in the pathogenesis of intestinal inflammation. Activated neutrophils infiltrate the mucosa and their products can be detected in feces. Numerous neutrophil derived proteins present in stool have been studied, including calprotectin, lactoferrin, and elastase. The most promising marker is calprotectin, because of its remarkable resistance to proteolytic degradation and its stability in stool kept at room temperature for at least seven days (Fagerhol (2000) Lancet, 356: 1783-1784). Calprotectin is a 36-kDa calcium- and zinc-binding protein complex that consists of one light and two heavy polypeptide chains (Dale et al. (1983) Eur. J. Biochem. 134: 1). It constitutes up to 60% of the cytosolic proteins in human neutrophil granulocytes (Johne et al. (1997) Mol. Pathol. 50: 113), and ileal tissue eosinophils. It is released during cell activation or cell death and has antiproliferative, antimicrobial, and immunomodulating functions (Fagerhol (2000) Lancet, 356: 1783-1784; Lundberg et al. (2005) Nat. Clin. Pract. Gastroenterol. Hepatol. 2: 96-102). Fecal calprotectin is nowadays used in clinical practice to evaluate disease activity in the follow-up of patients treated for active IBD and can be easily measured (Damms et al. (20-08) Int. J. Colorectal Dis. 23: 985-992).

Alpha-1-Antitrypsin (A1AT)

Alpha-1-Antitrypsin (A1AT) is a protease inhibitor that protects tissues from enzymes of inflammatory cells, especially neutrophil elastase. A1AT is one of the principal serum proteins and has a reference range in serum of ˜1.5-3.5 g/l, but the concentration can increase to a very high level during acute inflammation. A1AT is highly resistant to proteolysis in the intestine and can be excreted intact in the feces (see, e.g., Sharp (1976) Gastroenterology, 70: 611). A1AT can extravasate from serum into the gut in the condition of increased intestinal permeability, and finally be detected in the feces. This supports fecal A1AT as a biomarker of intestinal permeability. There are commercial kits to measure the fecal A1AT.

Intestinal Bacteria-Associated Markers

Lipopolysaccharide (LPS)

Several studies have successfully used LPS assays to show endotoxemia, mostly in patients with sepsis (see, e.g., Bates et al. (1998) AMCC Sepsis Project Working Group. Clin. Infect. Dis. 27: 582-591). Enhanced levels of LPS were found also in patients with obesity and metabolic syndrome (see, e.g., Bergheim et al. (2008) J. Hepatol. 48: 983-992; Thuy et al. (2008) J. Nutr. 138: 1452-1455), which is believed to indicate bacterial translocation from the gut lumen to the circulation as a consequence of intestinal barrier function failure. While LPS can be quite easily measured in portal vein blood in animals, it remains a challenge to measure LPS in peripheral blood in humans and it requires careful standardization of the measurement.

Circulating Endotoxin Core Antibodies (EndoCAb)

Measurement of circulating EndoCAb allowing the quantification of immunoglobulins (IgG, IgM and IgA) against the inner core of endotoxin have been proposed for the acute phase of intestinal barrier damage. This inner core consists of a hydrophobic part, lipid A, which is attached to a core oligosaccharide. Lipid A is highly conserved across the whole range of Gram-negative microbiota. Moreover, it is this part that is considered responsible for endotoxin toxicity. Several studies showed decreased EndoCAb levels postoperatively, accounting for the degree of exposure to endotoxin (see, e.g., Strutz et al. (1999) Intensive Care Med. 25: 435-444; Bennett-Guerrero et al. (2001) J. Cardiothorac. Vasc. Anesth. 15: 451-454). Thus, consumption of these circulating immunoglobulins following translocation of gut-derived endotoxins can be used to acquire indirect information on the intestinal epithelial barrier function.

Plasma D-Lactate Level

Plasma D-lactate levels have been used as a marker for diagnosis of bacterial infections, since D-lactate is a fermentation product produced by many bacteria including those present in the human gastrointestinal tract. Low circulating levels of D-lactate are found in healthy individuals, but in case of intestinal barrier function loss, these levels will rise as a consequence of increased translocation across the intestinal mucosa.

Various studies proposed a relationship between plasma D-lactate and intestinal permeability, e.g. in patients undergoing open aortic surgery and ischemic colonic injury.

Fecal Butyrate Concentrations

Generation of SCFA such as butyrate depends on prebiotic and other dietetic factors as well as on the composition and activity of the intestinal microbiota. It has been shown that butyrate decreases bacterial translocation in cells models (see, e.g., Lewis et al. (2010) Inflamm. Bowel Dis. 16: 1138-1148) and modifies the expression of the tight junction proteins claudin-1 and claudin-2 in favor of a barrier preservation (see, e.g., Ploger et al. (2012) Ann. N.Y. Acad. Sci. 1258: 52-59; Wang et al. (2012) Dig. Dis. Sci. 57: 3126-3135). Therefore, butyrate deficiency can be taken as an indirect indicator of impaired intestinal barrier function.

Other Markers.

Other markers of intestinal immunity such as secretory IgA (see, e.g., Lebreton et al. (2012) Gastroenterology, 143: 698-707) and defensins have been proposed as markers of intestinal permeability. Whereas secretory IgA has been examined in patients with celiac disease, defensins have been analyzed mostly in patients with IBD (see, e.g., Wehkamp et al. (2008) Mucosal Immunol. Suppl 1: S67-S74). More recently, fecal human β-defensin-2 has been suggested as a marker for intestinal permeability in neonates (see, e.g. Campeotto et al. (2010) Neonatology, 98: 365-369).

The foregoing method of assessing intestinal permeability are illustrative and non-limiting. Using the teachings provided herein numerous other permeability assays will be available to one of skill in the art.

### Pharmaceutical Formulations.

In various embodiments the agent(s) that inhibit superoxide (or hydrogen peroxide) production from the outer ubiquinone-binding site of complex III of the mitochondrial electron transport chain (site III_{Qo}) (S3QELs) are administered as a pharmaceutical formulation.

In various embodiments the agent(s) described herein (e.g., S3QELs) can be administered in the “native” form or, if desired, in the form of salts, esters, amides, derivatives, and the like, provided the salt, ester, amide, or derivative is suitable pharmacologically, e.g., effective in the present method(s). Salts, esters, amides, and other derivatives of the agent(s) described herein vehicles can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, a pharmaceutically acceptable salt can be prepared for any compound described herein having a functionality capable of forming a salt (e.g., such as a carboxylic acid functionality of the compounds described herein). A pharmaceutically acceptable salt is any salt that retains the activity of the parent compound and does not impart any deleterious or untoward effect on the subject to which it is administered and in the context in which it is administered.

Methods of pharmaceutically formulating the compounds described herein as salts, esters, amides, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the compounds described herein can include halide salts, such as may be prepared using hydrochloric or hydrobromic acids. Conversely, preparation of basic salts of the agent(s) described herein can be prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

In various embodiments, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, formate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, e.g., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the compounds identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., amyloidogenic pathologies).

The active agent(s) described herein can also be combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the agent(s), or excipients or other stabilizers and/or buffers.

Other physiologically acceptable compounds, particularly of use in the preparation of tablets, capsules, gel caps, and the like include, but are not limited to binders, diluent/fillers, disentegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g., calcium carbonate, carboxymethylcellulose calcium, sodium starch glycollate, crospovidone etc.), a binder (e.g., alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the agent(s) described herein (S3QELs) and the resulting composition is compressed. Where necessary the compressed product is coated, e.g., known methods for masking the taste or for enteric dissolution or sustained release. Suitable coating materials include, but are not limited to ethyl-cellulose, hydroxymethylcellulose, polyoxyethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the agent(s) described herein and on the particular physio-chemical characteristics of the agent(s).

In certain embodiments, the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectable, implantable sustained-release formulations, mucoadherent films, topical varnishes, lipid complexes, etc.

Pharmaceutical compositions comprising the agent(s) described herein (e.g., S3QELs) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

Systemic formulations include, but are not limited to, those designed for administration by injection, e.g., subcutaneous, intravenous, intramuscular, intrathecal or intraperitoneal injection, as well as those designed for transdermal, transmucosal oral or pulmonary administration. For injection, the agent(s) described herein can be formulated in aqueous solutions, preferably in physiologically compatible buffers such as Hanks solution, Ringer's solution, or physiological saline buffer and/or in certain emulsion formulations. The solution can contain formulatory agents such as suspending, stabilizing and/or dispersing agents. In certain embodiments, the agent(s) described herein (S3QELs) can be provided in powder form for constitution with a suitable vehicle, e.g., sterile pyrogen-free water, before use. For transmucosal administration, penetrants appropriate to the barrier to be permeated can be used in the formulation. Such penetrants are generally known in the art.

For oral administration, the compounds can be readily formulated by combining the agent(s) described herein (e.g., S3QELs) with pharmaceutically acceptable carriers well known in the art. Such carriers enable the compounds described herein to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added. For buccal administration, the compositions may take the form of tablets, lozenges, etc. formulated in conventional manner.

For administration by inhalation, the agent(s) described herein (S3QELs) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In various embodiments, the agent(s) described herein can be formulated in rectal or vaginal compositions such as suppositories or retention enemas, e.g., containing conventional suppository bases such as cocoa butter or other glycerides.

In addition to the formulations described previously, the agent(s) described herein may also be formulated as a depot preparation. Such long acting formulations can be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to protect and deliver pharmaceutically active compounds. Certain organic solvents such as dimethylsulfoxide also can be employed, although usually at the cost of greater toxicity. Additionally, the compounds may be delivered using a sustained-release system, such as semipermeable matrices of solid polymers containing the therapeutic agent. Various uses of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

In certain embodiments, the agent(s) described herein (S3QELs) and/or formulations thereof are administered orally. This is readily accomplished by the use of tablets, caplets, lozenges, liquids, and the like. In various embodiments, the compound(s) in the oral compositions can be either coated or non-coated. The preparation of enteric-coated particles is disclosed for example in U.S. Pat. Nos. 4,786,505 and 4,853,230.

In certain embodiments, the agent(s) described herein (S3QELs) and/or formulations thereof are administered systemically (e.g., orally, or as an injectable) in accordance with standard methods well known to those of skill in the art. In other embodiments, the agents can also be delivered through the skin using conventional transdermal drug delivery systems, e.g., transdermal “patches” wherein the compound(s) and/or formulations described herein are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

In certain embodiments, one or more agent(s) described herein can be provided as a “concentrate”, e.g., in a storage container (e.g., in a premeasured volume) ready for dilution, or in a soluble capsule ready for addition to a volume of water, alcohol, hydrogen peroxide, or other diluent.

In various embodiments, compositions contemplated herein typically comprise one or more of the various agent(s) described herein (S3QELs) in an effective amount to achieve a pharmacological effect or therapeutic improvement without undue adverse side effects. Illustrative pharmacological effects or therapeutic improvements include, but are not limited to a reduction in intestinal permeability and/or an amelioration of one or more symptoms of a pathology associated with abnormal intestinal permeability.

In various embodiments, the typical daily dose of agent(s) described herein (S3QELs) varies and will depend on various factors such as the individual requirements of the patients and the disease to be diagnosed and/or treated. In general, the daily dose of compounds can be in the range of 1-1,000 mg or 1-800 mg, or 1-600 mg, or 1-500 mg, or 1-400 mg. In one illustrative embodiment a standard approximate amount of the agent(s) described herein (S3QELs) present in the composition can be typically about 1 to 1,000 mg, more preferably about 5 to 500 mg, and most preferably about 10 to 100 mg. In certain embodiments the agent(s) are administered only once, or for follow-up as required. In certain embodiments the agent(s) are administered once a day, in certain embodiments, administered twice a day, in certain embodiments, administered 3 times/day, and in certain embodiments, administered 4, or 6, or 6 or 7, or 8 times/day.

In certain embodiments the active agent(s) described herein are formulated in a single oral dosage form containing all active ingredients. Such oral formulations include solid and liquid forms. It is noted that solid formulations typically provide improved stability as compared to liquid formulations and can often afford better patient compliance.

In one illustrative embodiment, the one or more of agent(s) described herein (e.g., S3QELs) are formulated in a single solid dosage form such as single- or multi-layered tablets, suspension tablets, effervescent tablets, powder, pellets, granules or capsules comprising multiple beads as well as a capsule within a capsule or a double chambered capsule. In another embodiment, the agent(s) described herein (e.g., S3QELs) may be formulated in a single liquid dosage form such as suspension containing all active ingredients or dry suspension to be reconstituted prior to use.

In certain embodiments, the agent(s) described herein (e.g., S3QELs) are formulated as enteric-coated delayed-release granules or as granules coated with non-enteric time-dependent release polymers in order to avoid contact with the gastric juice. Non-limiting examples of suitable pH-dependent enteric-coated polymers are: cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. A suitable commercially available enteric material, for example, is sold under the trademark EUDRAGIT L 100-55®. This coating can be spray coated onto a substrate.

Illustrative non-enteric-coated time-dependent release polymers include, for example, one or more polymers that swell in the stomach via the absorption of water from the gastric fluid, thereby increasing the size of the particles to create thick coating layer. The time-dependent release coating generally possesses erosion and/or diffusion properties that are independent of the pH of the external aqueous medium. Thus, the active ingredient is slowly released from the particles by diffusion or following slow erosion of the particles in the stomach.

Illustrative non-enteric time-dependent release coatings are for example: film-forming compounds such as cellulosic derivatives, such as methylcellulose, hydroxypropyl methylcellulose (HPMC), hydroxyethylcellulose, and/or acrylic polymers including the non-enteric forms of the EUDRAGIT® brand polymers. Other film-forming materials can be used alone or in combination with each other or with the ones listed above. These other film forming materials generally include, for example, poly(vinylpyrrolidone), Zein, poly(ethylene glycol), poly(ethylene oxide), poly(vinyl alcohol), poly(vinyl acetate), and ethyl cellulose, as well as other pharmaceutically acceptable hydrophilic and hydrophobic film-forming materials. These film-forming materials may be applied to the substrate cores using water as the vehicle or, alternatively, a solvent system. Hydro-alcoholic systems may also be employed to serve as a vehicle for film formation.

Other materials suitable for making the time-dependent release coating of the compounds described herein include, by way of example and without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as hydroxymethylcellulose, hydroxyethylcellulose, and hydroxypropylcellulose; synthetic water-soluble cellulose-based lamina formers such as methyl cellulose and its hydroxyalkyl methylcellulose cellulose derivatives such as a member selected from the group consisting of hydroxyethyl methylcellulose, hydroxypropyl methylcellulose, and hydroxybutyl methylcellulose; other cellulose polymers such as sodium carboxymethylcellulose; and other materials known to those of ordinary skill in the art. Other lamina forming materials that can be used for this purpose include, but are not limited to poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

While the agent(s) described herein (e.g., S3QELs) and methods of use thereof are described herein with respect to use in humans, they are also suitable for animal, e.g., veterinary use. Thus certain illustrative organisms include, but are not limited to humans, non-human primates, canines, equines, felines, porcines, ungulates, largomorphs, and the like.

The foregoing formulations and administration methods are intended to be illustrative and not limiting. It will be appreciated that, using the teaching provided herein, other suitable formulations and modes of administration can be readily devised.

### Kits.

In various embodiments the agent(s) described herein (e.g., S3QELs) can be provided in kits. In certain embodiments the kits comprise the agent(s) described herein enclosed in multiple or single dose containers. In certain embodiments the kits can comprise component parts that can be assembled for use. For example, one or more S3QELs in lyophilized form and a suitable diluent may be provided as separated components for combination prior to use. In certain embodiments a kit may include one or more agent(s) described herein (e.g., S3QELs) and a second therapeutic agent for co-administration. The active agent and second therapeutic agent may be provided as separate component parts. A kit may include a plurality of containers, each container holding one or more unit dose of the agent(s) described herein (e.g., S3QELs). The containers are preferably adapted for the desired mode of administration, including, but not limited to tablets, gel capsules, sustained-release capsules, and the like for oral administration; depot products, pre-filled syringes, ampules, vials, and the like for parenteral administration; and patches, and the like for topical administration, e.g., as described herein.

In certain embodiments the kits can further comprise instructional materials. In certain embodiments the informational material(s) indicate that the administering of the compositions can result in adverse reactions including but not limited to allergic reactions such as, for example, anaphylaxis. The informational material can indicate that allergic reactions may exhibit only as mild pruritic rashes or may be severe and include erythroderma, vasculitis, anaphylaxis, Steven-Johnson syndrome, and the like. In certain embodiments the informational material(s) may indicate that anaphylaxis can be fatal and may occur when any foreign substance is introduced into the body. In certain embodiments the informational material may indicate that these allergic reactions can manifest themselves as urticaria or a rash and develop into lethal systemic reactions and can occur soon after exposure such as, for example, within 10 minutes. The informational material can further indicate that an allergic reaction may cause a subject to experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, eosinophilia, or a combination thereof.

In certain embodiments the instructional materials describe the use of the active agent(s) described herein in a method for the treatment or prophylaxis of an age-related and/or pathology-associated increase in intestinal barrier permeability.

While the instructional materials typically comprise written or printed materials they are not limited to such. Any medium capable of storing such instructions and communicating them to an end user is contemplated herein. Such media include, but are not limited to electronic storage media (e.g., magnetic discs, tapes, cartridges, chips), optical media (e.g., CD ROM), and the like. Such media may include addresses to internet sites that provide such instructional materials.

In some embodiments, the kits can comprise one or more packaging materials such as, for example, a box, bottle, tube, vial, container, sprayer, insufflator, intravenous (I.V.) bag, envelope, and the like, and at least one unit dosage form of an agent comprising active agent(s) described herein and a packaging material. In some embodiments, the kits also include instructions for using the composition as prophylactic, therapeutic, or ameliorative treatment for the disease of concern.

### EXAMPLES

The following examples are offered to illustrate, but not to limit the claimed invention.

### Example 1

### Mitochondrial Superoxide from Complex III Drives Diet-Induced Intestinal Barrier Dysfunction

The intestinal epithelium has several barriers, consisting of a mucous layer, tight junctions between cells, and a substantial set of resident immune cells, that protect the host from pathogens and toxins in the gut lumen (Peterson & Artis (2014) Nat. Rev. Immunol. 14:141-153; Vighi et al. (2008) Clin. Exp. Immunol. 153(Suppl 1): 3-6). Disruption of the intestinal epithelial barrier permits the passage of these pathogens and toxins, which can initiate and exacerbate disease and aging (Fink & Delude (2005) Crit. Care Clin. 21: 177-196; Doig et al. (1998) Am. J. Respir. Crit. Care Med. 158: 444-451; Harris et al. (1992) Intensive Care Med. 18: 38-41). Understanding and preventing the underlying causes of intestinal barrier decline can help prevent disease and possibly slow aging (Fasano & Shea-Donohue (2005) Nat. Clin. Pract. Gastroenterol. Hepatol. 2: 416-422; Farhadi et al. (2003) J. Gastroenterol. Hepatol. 18: 479-497; König et al. (2016) Clin. Transl. Gastroenterol. 7: e196; Odenwald & Turner (2017) Nat. Rev. Gastroenterol. Hepatol. 14: 9-21). Oxidative stress has been argued to be an important driver of aging and age-related pathologies including intestinal barrier dysfunction (Liguori et al. (2018) Clin. Interv. Aging 13: 757-772; Tian et al. (2017) Oxid. Med. Cell. Longev. 2017, U.S. Pat. No. 4,535,194; Hale et al. (2012) PLoS One 7: e41797; Wang et al. (2014) Am. J. Pathol. 184: 2516-2527).

In the present study we investigated whether mitochondrial superoxide generated by complex III of the mitochondrial electron transport chain is a cause of intestinal barrier disruption, using Drosophila and mice fed high-nutrient diets as models of accelerated metabolic disease and aging.

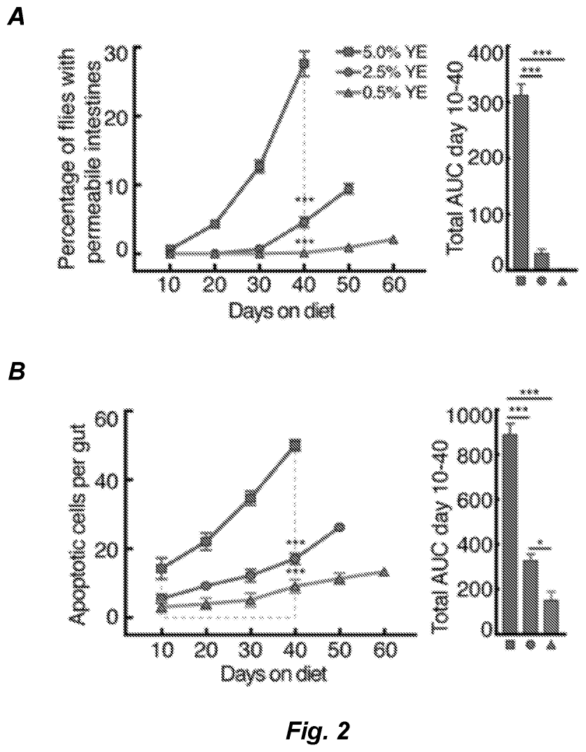

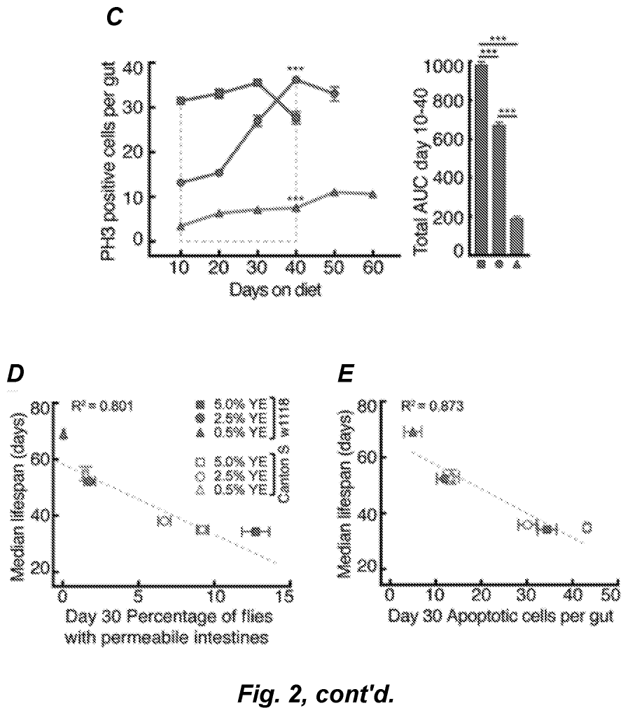

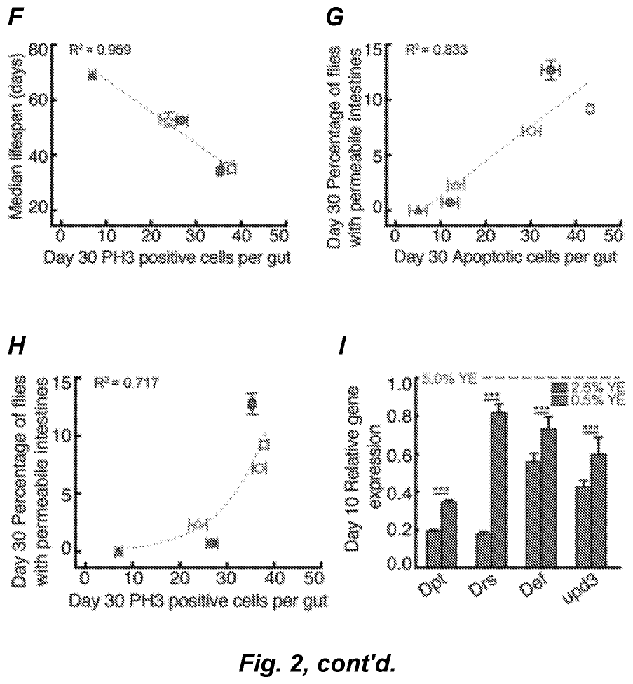

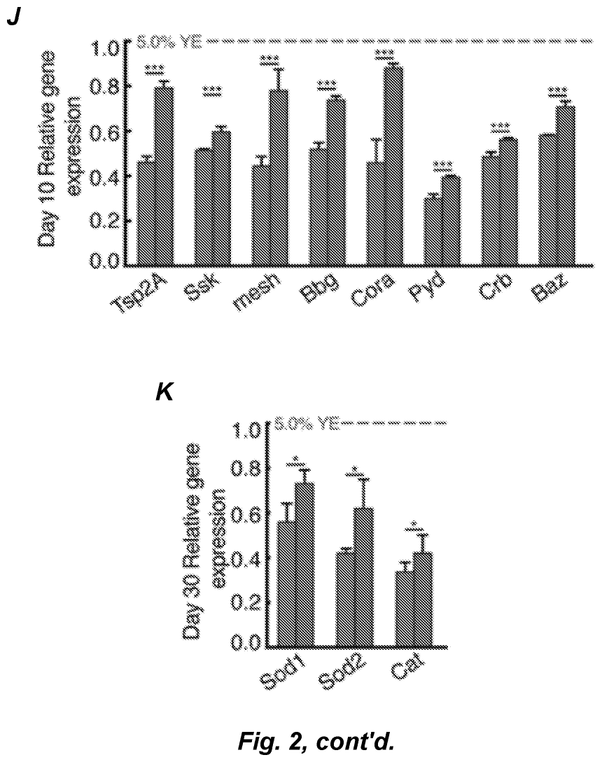

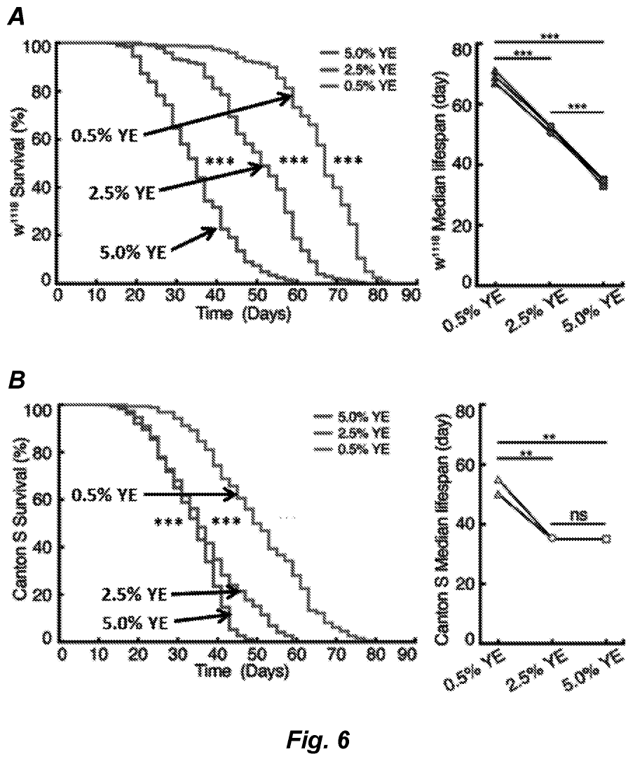

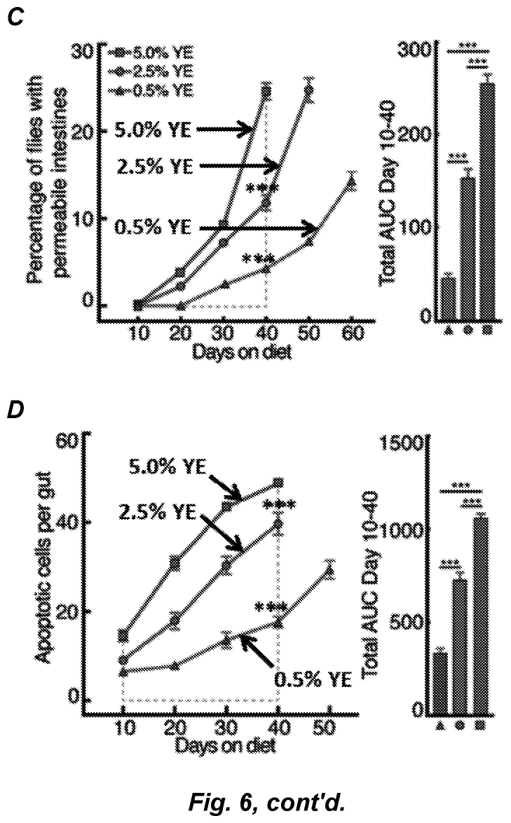

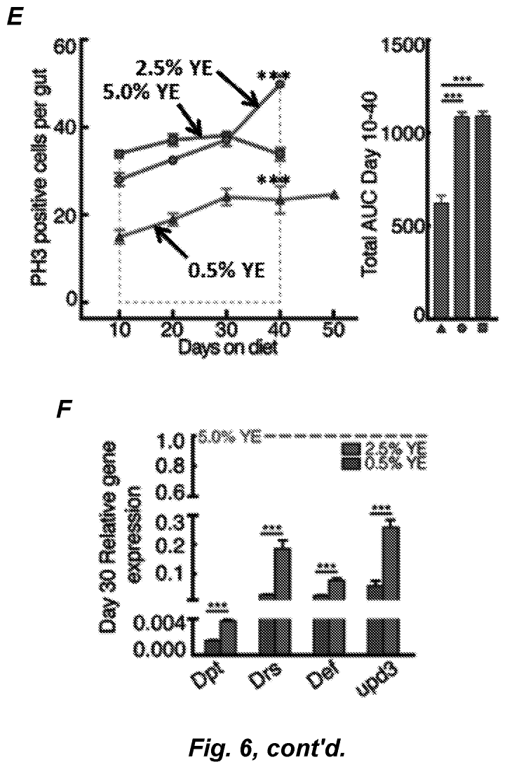

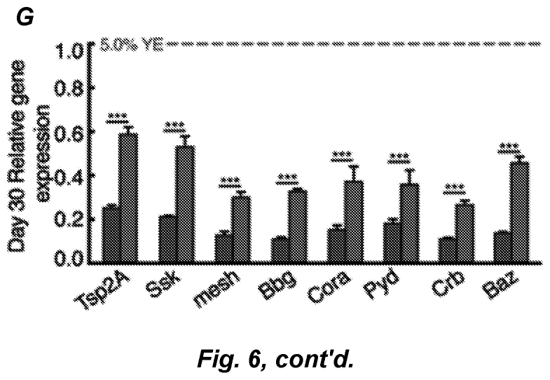

In many species, restricted or ad-libitum feeding impacts both healthspan and lifespan. In Drosophila, decreasing the amount of dietary protein (in the form of yeast extract, YE) below the conventional 2.5% is known to increase median lifespan, whereas increasing dietary YE content decreases median lifespan (Chapman & Partridge (1996) Proc. R. Soc. B Biol. Sci. 263: 755-759). This was confirmed in the two Drosophila strains (w¹¹¹⁸ and Canton S) used here (FIG. 6, panels A, B). We measured intestinal permeability of flies by feeding a blue food dye that is normally not absorbed; flies with permeable intestinal epithelium become blue (Rera et al. (2012) Proc. Natl. Acad. Sci. USA, 109: 21528-21533). The percentage of blue flies was higher at greater YE % in both w¹¹¹⁸ (FIG. 2, panel A) and Canton S flies (FIG. 6, panel C). Further analysis revealed that median lifespan was decreased in flies of either strain when the incidence of intestinal permeability was enhanced by greater dietary YE % (FIG. 2, panel D), suggesting that intestinal permeability influences lifespan.

There is a critical balance between cell proliferation and apoptosis in the Drosophila intestine, and tipping this balance can be detrimental (Liang et al. (2017) Nature 548: 588-591). Enterocyte damage can cause apoptosis and therefore trigger proliferation of intestinal cells for repair and maintenance of tissue integrity (Ohlstein & Spradling (2006) Nature 439: 470-474; Amcheslavsky & Ip (2009) Cell Stem Cell 4: 49-61). Feeding higher YE % to w¹¹¹⁸ and Canton S flies increased both the number of apoptotic cells and the number of proliferating (PH3 positive) cells per intestine (FIG. 2, panels B, C; FIG. 6, panels D, E), supporting this view. Apoptotic and PH3-positive intestinal cell number correlated negatively with median lifespan (FIG. 2, panels E, F) and positively with intestinal permeability (FIG. 2, panels G, H).

We confirmed an increase in intestinal permeability using known gene expression hallmarks of a disrupted intestinal barrier. Antimicrobial peptides (AMPs) are expressed in response to intestinal damage and infection, and the Upd3 cytokine is released upon enterocyte damage (Lucchetta & Ohlstein (2012) Wiley Interdiscip. Rev. Dev. Biol. 1: 781-788). The intestinal expression of the AMP genes Dpt, Drs and Def, and the upd3 gene increased at higher YE % on day 10 (FIG. 2, panel I) and day 30 (FIG. 6, panel F) after the diet switch. The intestinal expression of septate junction genes (the equivalent of tight junctions in vertebrates) also increased at higher dietary YE % (FIG. 2, panel J; FIG. 6, panel G). Elevated expression of tight junction genes in flies fed a high-nutrient diet suggests a response to epithelial tight junction damage that is related to an increase in intestinal permeability.

Next, we investigated the mechanism by which a rich diet decreased median lifespan and increased intestinal permeability. Increased oxidative stress is reported in intestines of animals fed high-nutrient diets (Lee et al. (2017) Diabetes 66: 2072-2081; Paglialunga et al. (2015) Diabetologia 58: 1071-1080; Patel et al. (2007) J. Clin. Endocrinol. Metab. 92: 4476-4479; Feillet-Coudray et al. (2014) Free Radic. Res. 48: 1232-1246). We hypothesized that mitochondrial production of reactive oxygen species might drive increased intestinal permeability, a suggestion supported by the observation of elevated intestinal gene expression of superoxide dismutases (which dismutate superoxide to hydrogen peroxide) in the cytosol (Sod1) and mitochondria (Sod2), and of catalase (Cat), which reduces hydrogen peroxide to water, in flies fed higher YE % (FIG. 2, panel K).

The outer ubiquinone-binding site of complex III of the mitochondrial electron transport chain (site III_{Qo}) has the largest capacity of all mitochondrial sites to produce superoxide, which it delivers into both the mitochondrial matrix and the cytosol (Brand (2016) Free Radic. Biol. Med. 100: 14-31; St-Pierre et al. (2002) J. Biol. Chem. 277: 44784-44790). To test whether superoxide produced by site III_{Qo} causes intestinal permeability, flies fed a 5% YE diet were also fed S3QELs, which are small molecules that suppress superoxide production from site III_{Qo} without affecting energy metabolism (Orr et al. (2015) Nat. Chem. Biol. 11: 834-836). We tested three structurally different S3QELs: S3QEL1.2, S3QEL2.2, and S3QEL3. These act as their own internal control as all three S3QELs should give the same response if their effect is on-target, but different responses if it is off-target.

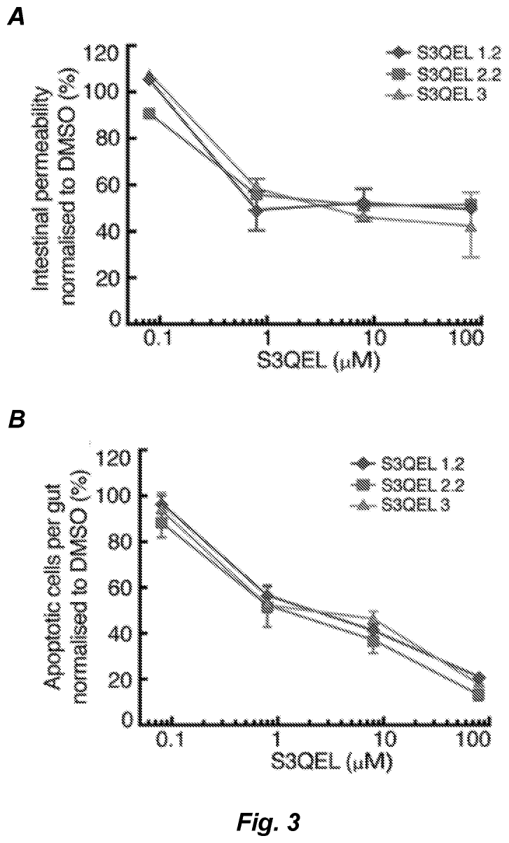

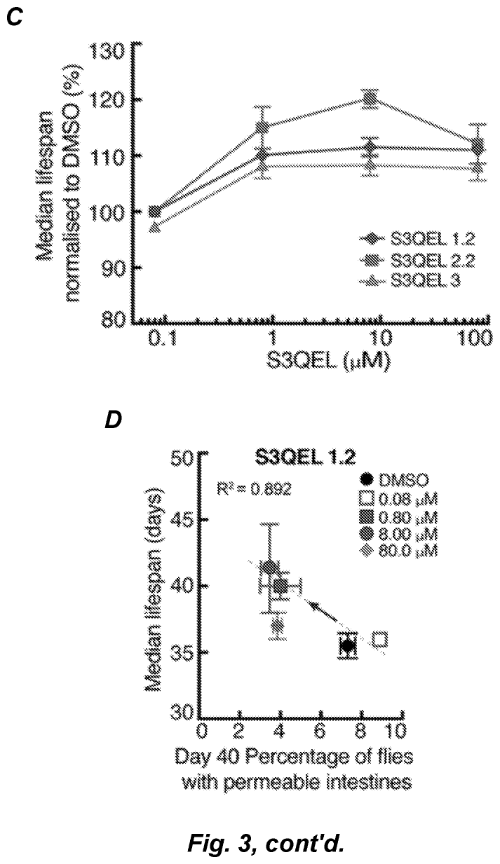

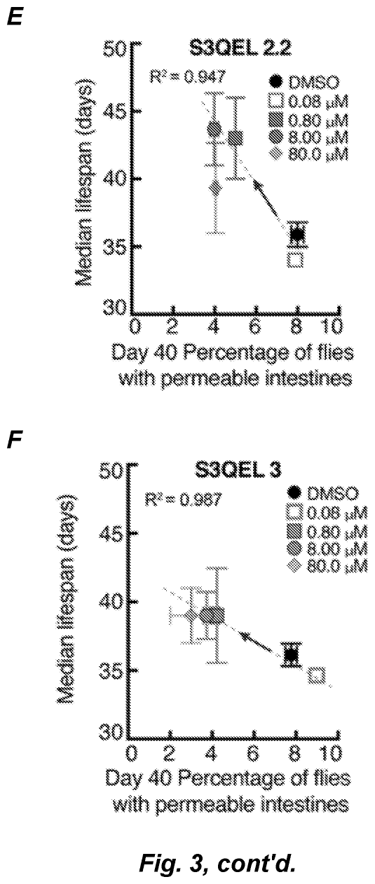

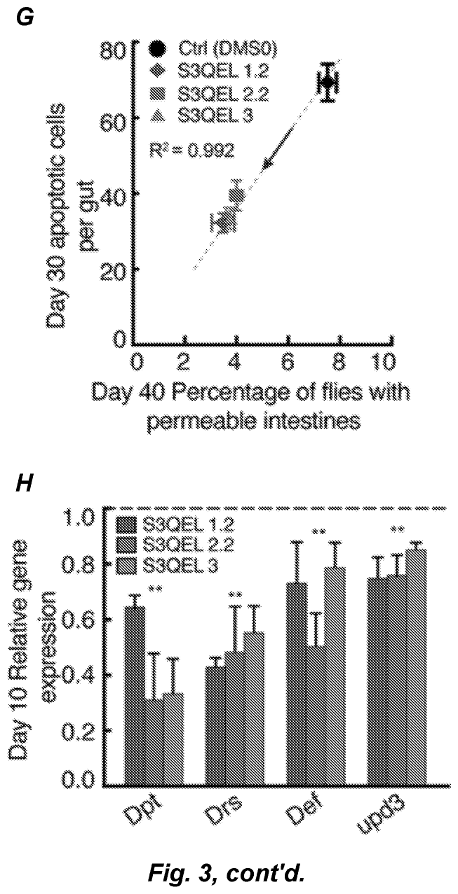

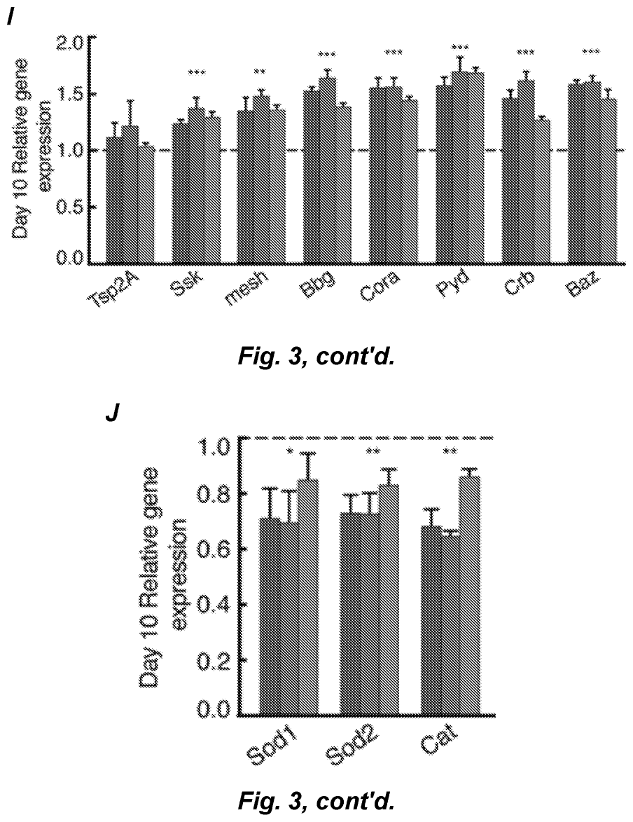

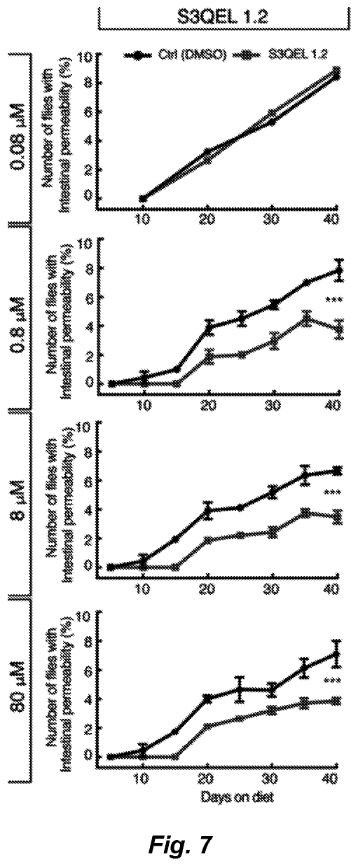

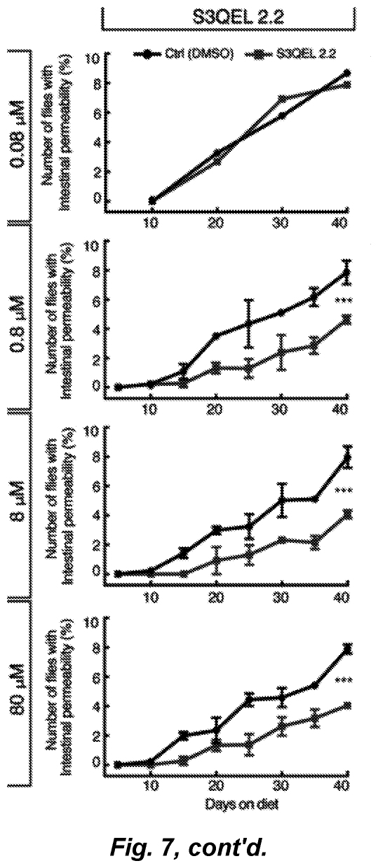

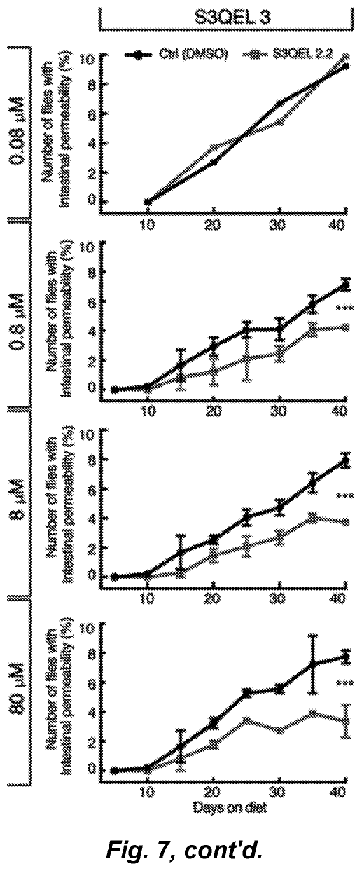

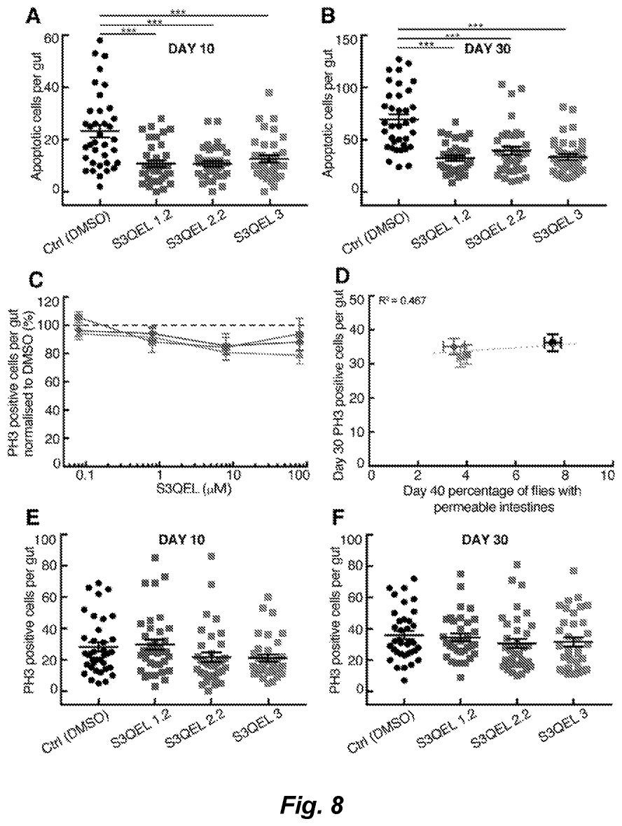

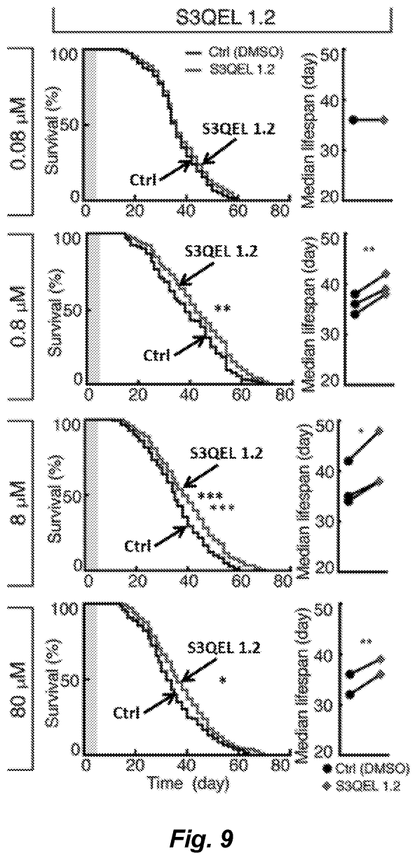

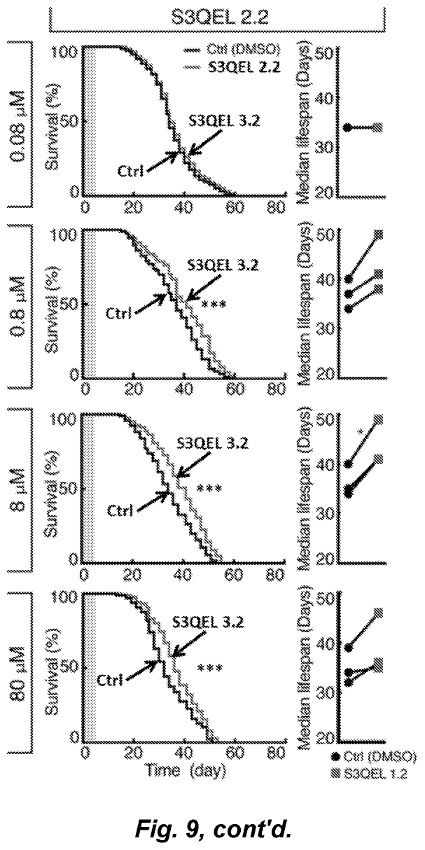

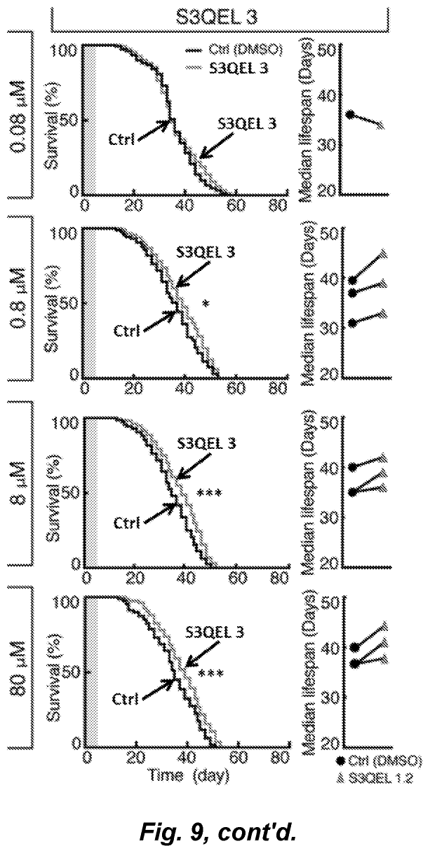

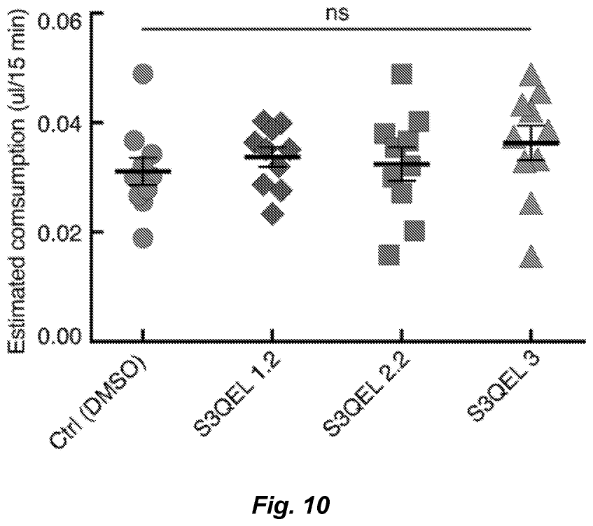

FIG. 3, panel A and FIG. 7 show that the incidence of intestinal permeability in w¹¹¹⁸ Drosophila fed a 5% YE diet was approximately halved by co-feeding each of the three S3QELs. The S3QELs also decreased the number of apoptotic cells per intestine (FIG. 3, panel B) (FIG. 8, panels A, B show that 8 μM S3QELs halved apoptotic cell number at both day 10 and day 30), and increased median lifespan by 10-20% (FIG. 3, panel C; FIG. 9). Interestingly, S3QELs had no significant effect on the intestinal incidence of PH3-positive cells (FIG. 8, panels C-F). Further analysis revealed that median lifespan was greater (FIG. 3, panels D-F) and apoptotic cell number was smaller (FIG. 3, panel G) when feeding S3QELs at concentrations greater than 0.08 μM decreased the incidence of intestinal permeability. These data suggest that by inhibiting superoxide production from site III_{Qo}, S3QELs decrease intestinal permeability, which in turn increases median lifespan. S3QELs did not decrease food consumption (FIG. 10), ruling out the possibility that they worked by mimicking caloric restriction. In contrast to the effects of S3QELs, feeding of S1QEL1.2 or S1QEL2.1 (suppressors of site I_{Q} electron leak (Brand et al. (2016) Cell Metab. 24: 582-592)) did not protect against induction of intestinal permeability (preliminary data, not shown). Note that both S1QELs and S3QELs suppress superoxide/hydrogen peroxide production by mitochondria isolated from Drosophila (Id.).

Feeding S3QELs decreased the expression of the intestinal damage and inflammatory gene markers (FIG. 3, panel H), supporting the conclusion of a decrease in intestinal permeability relative to 5% YE diet. Expression of the tight-junction genes did not decrease but trended upwards (FIG. 3, panel I). Feeding S3QELs decreased the expression of the antioxidant genes (FIG. 3, panel J), consistent with a decrease in oxidative stress.

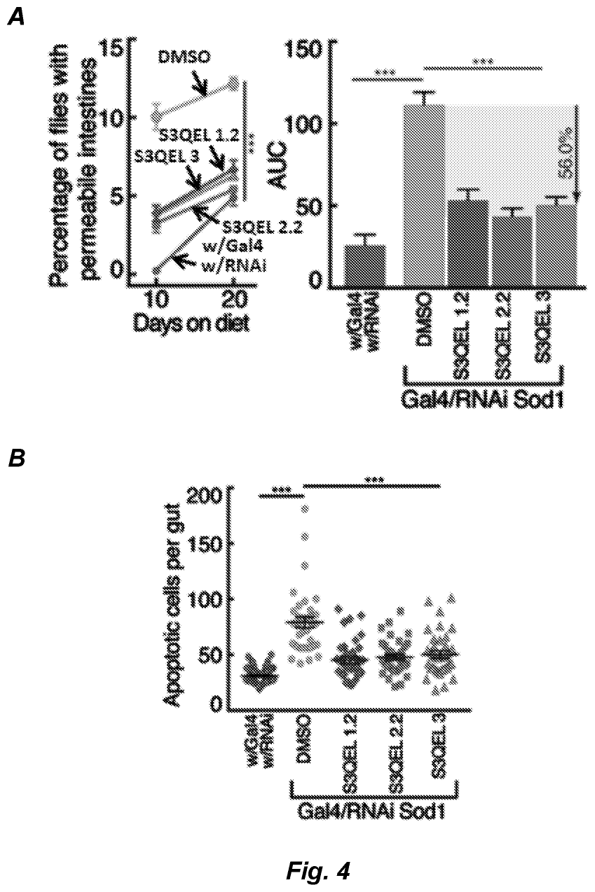

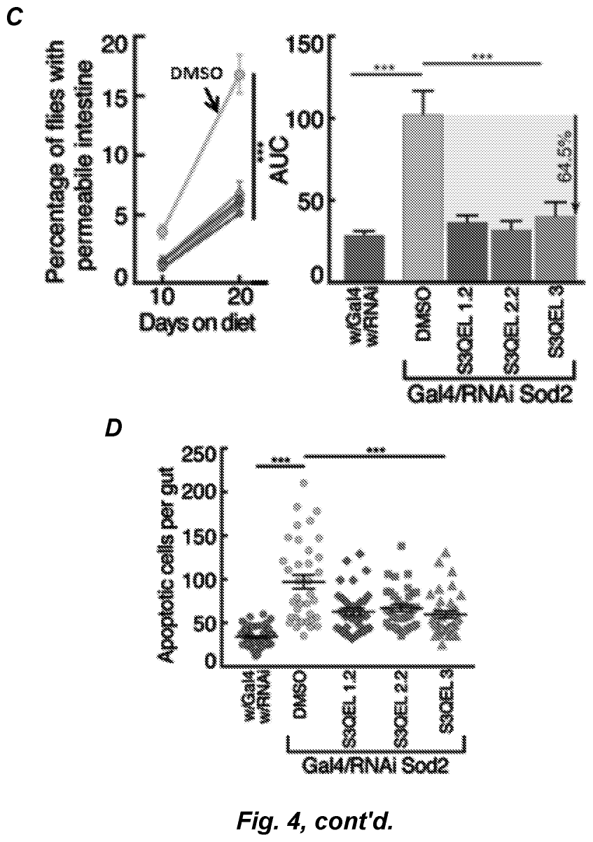

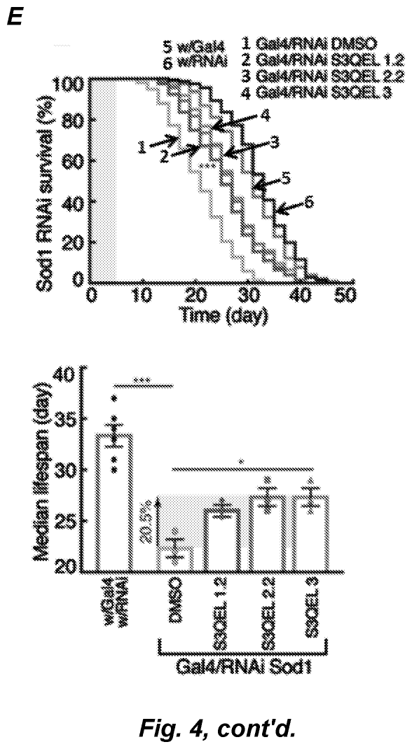

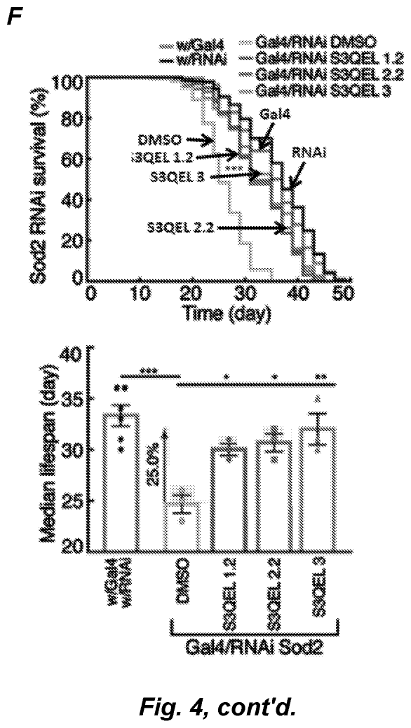

We investigated whether the effects of S3QELs were exerted specifically within gut enterocytes, which will be more readily exposed to S3QELs from the gut lumen, rather than a systemic effect. Using the NP1-GAL4 driver, we used RNAi to knock down expression of Sod1 and Sod2 specifically in intestinal enterocytes to decrease cytosolic and matrix superoxide removal and thereby increase superoxide levels in the two compartments in these cells. FIG. 4 shows that each knockdown increased the incidence of intestinal permeability and intestinal apoptotic cell number and decreased median lifespan, showing that raised superoxide levels just in enterocytes can drive these phenotypes. After either Sod1 or Sod2 knockdown, S3QELs protected against the induced intestinal permeability (FIG. 4, panels A, C), increase in intestinal apoptotic cell number (FIG. 4, panels B, D) and decrease in median lifespan (FIG. 4, panels E, F). These results support the idea that, by decreasing superoxide production from complex III into the cytosol and matrix, S3QELs can work specifically in enterocytes to decrease superoxide production to improve intestinal homeostasis and extend lifespan.

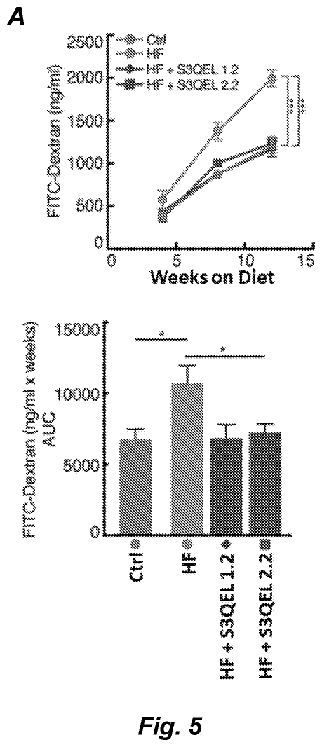

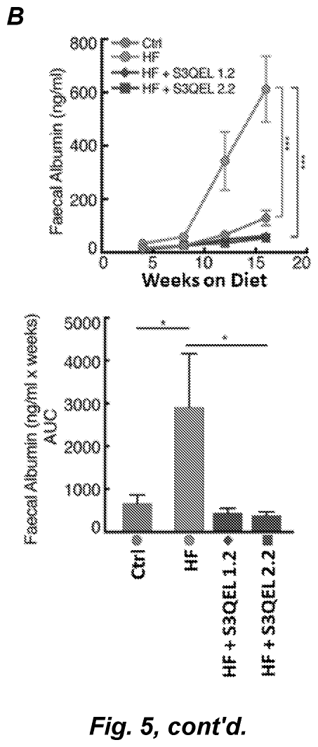

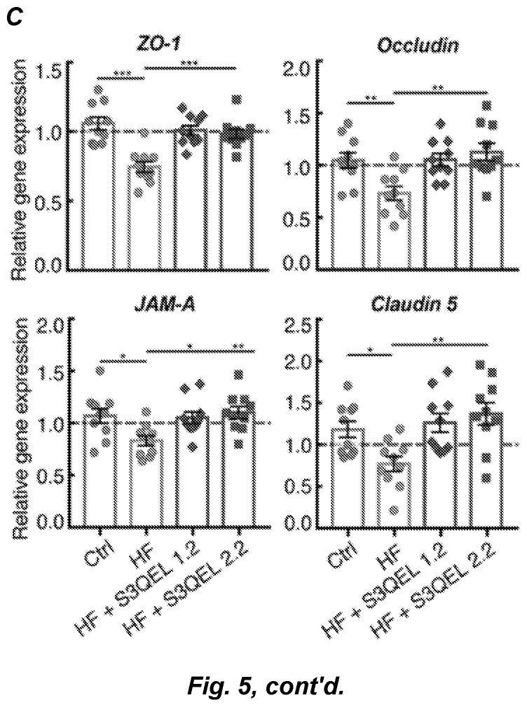

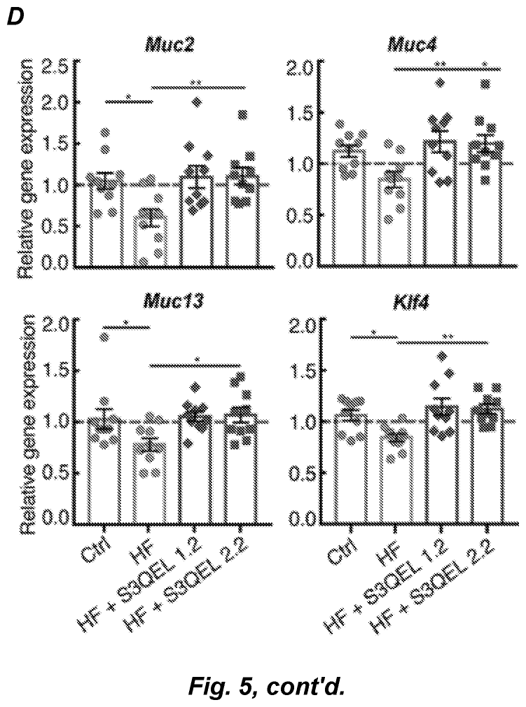

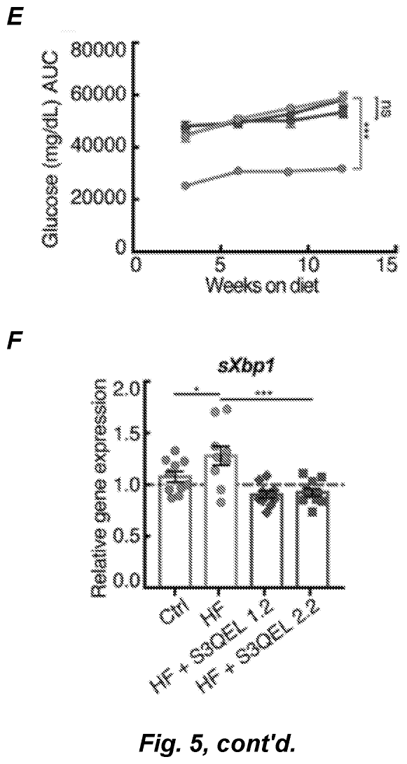

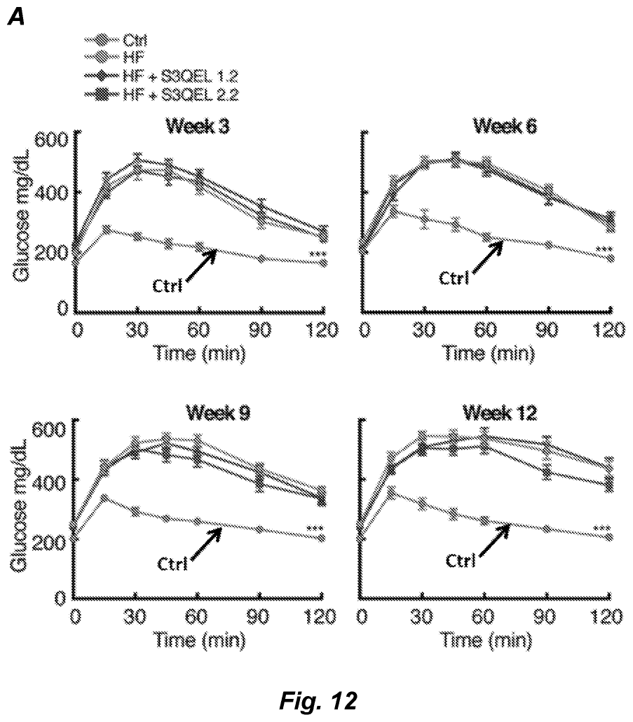

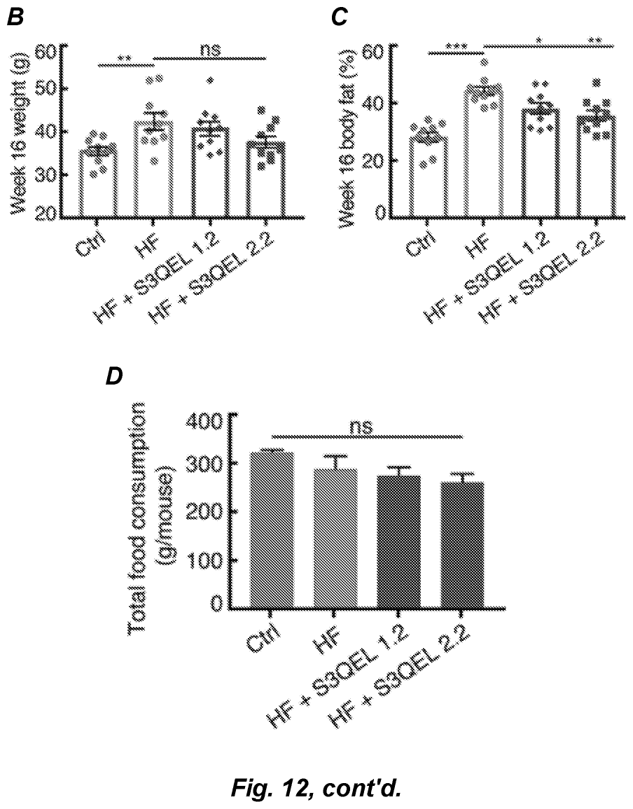

To examine whether the effects of S3QELs in Drosophila are conserved, we tested whether they protect against intestinal permeability in a mouse model. To draw parallels to Drosophila, we tested oral delivery of S3QELs in mice fed a high-fat diet (60% kcal). High-fat feeding has been shown to increase oxidative stress and induce intestinal permeability in mice (Ahmad et al. (2017) Sci. Rep. 7: 5125; Murakami et al. (2016) J. Food Sci. 81: H216-H222). We found that high-fat feeding induced intestinal permeability in mice (measured both by the uptake of FITC-dextran from the gut into blood plasma (FIG. 5, panel A) and by the appearance of plasma-derived albumin in feces (FIG. 5, panel B) and decreased the expression of tight-junction (FIG. 5, panel C) and mucin genes (FIG. 5, panel D). It also induced glucose intolerance (FIG. 5, panel E; FIG. 12, panel A), increased body weight and adiposity (FIG. 12, panels B, C) and increased the expression of an ER-stress gene (FIG. 5, panel F).

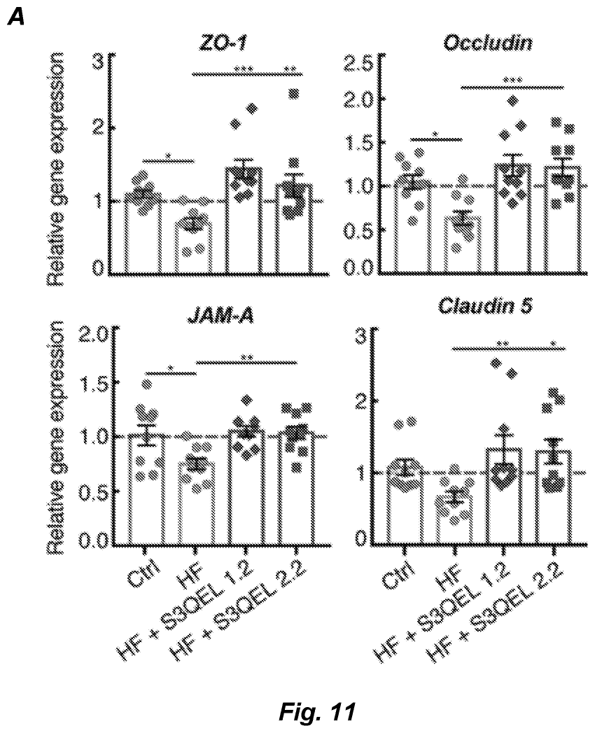

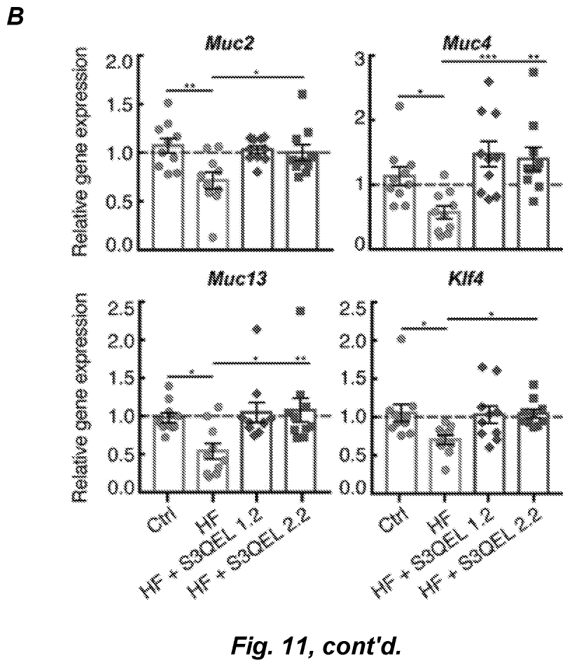

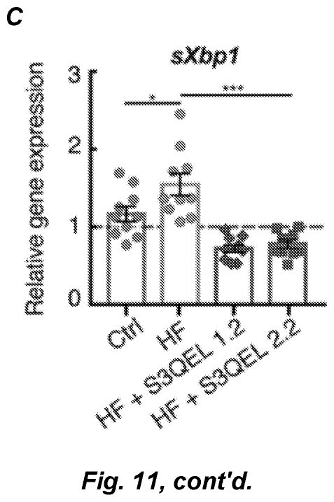

S3QEL1.2 and S3QEL2.2 strongly protected against the increases in intestinal permeability in mice (FIG. 5, panels A, B). They protected against the decrease in tight-junction and mucin gene expression in both colon and distal small intestine (FIG. 5, panels C, D; FIG. 11). Expression of the goblet cell differentiation transcription factor Klf4 is known to be decreased upon high-fat feeding, and this decrease is one cause of decreased mucin expression (Gulhane et al. (2016) Sci. Rep. 6: 28990). Klf4 expression was decreased by high-fat feeding and protected by S3QELs (FIG. 5, panel D). Together, these results strongly suggest that superoxide production from mitochondrial complex III drives intestinal permeability in mice as it does in Drosophila.

Treatment with S3QELs did not improve glucose tolerance in mice fed the high-fat diet (FIG. 5, panel E; FIG. 12, panel A). Weight gain (FIG. 12, panel B), body fat (FIG. 12, panel C) and food consumption (FIG. 12, panel D) of mice fed the high-fat diet each trended downwards with S3QEL feeding, but only the decrease in body fat reached statistical significance. Thaiss and colleagues have proposed that the hyperglycemia induced by a high-fat diet drives intestinal permeability (Thaiss et al. (2018) Science 359: 1376-1383). The lack of significant effect of S3QELs on glucose tolerance despite the improvement in intestinal permeability suggests that hyperglycemia is upstream of intestinal permeability and supports this model (Thaiss et al. (2018) Science 359: 1376-1383) over the alternative model that increased gut permeability drives glucose intolerance. We suggest that high-fat diet and the resulting hyperglycemia increase superoxide production from site III_{Qo} of the mitochondrial electron transport chain in gut epithelial cells, which in turn drives ER stress, increased intestinal permeability, and other phenotypes. This hypothesis explains why treatment with S3QELs protects against ER stress (FIG. 5, panel F) and intestinal permeability (FIG. 5, panels A, B), but not against impaired glucose tolerance (FIG. 5, panel E; FIG. 12, panel A).

We conclude that superoxide production from site III_{Qo} is necessary (and, in flies, raising superoxide levels by Sod knockdown in enterocytes is sufficient) for the development of diet-induced intestinal barrier dysfunction in flies and mice. Suppressing superoxide production by site III_{Qo} in complex III of the mitochondrial electron transport chain using S3QELs has potential therapeutic value in intestinal and other diseases of aging.

### Supplementary Materials:

Methods and Materials

Experimental Design:

Experiments were designed to examine the effects of suppressing the production of superoxide by complex III using S3QELs on intestinal permeability in Drosophila and mice.

Fly Strains and Husbandry:

Control lines were w¹¹¹⁸ and Canton S. The GAL4 driver line was NP1-GAL4; tub-GAL80ts (67067) from Bloomington Drosophila Stock Center. The UAS responder lines were Sod1-RNAi (v108307) and Sod2-RNAi (v42162) from Vienna Drosophila Resource Center (VDRC). All fly lines were maintained on standard yeast medium containing (w/v) 1.5% dry active yeast (Saf-instant), 5% sucrose, 0.46% agar, 8.5% cornmeal and 1% v/v acid mix (a 1:1 v/v mix of 83.6% w/v orthophosphoric acid and 10% w/v propionic acid) in distilled water. The mixture was boiled with continuous stirring for 10 min and allowed to cool, then the acid mix was stirred in. The yeast medium was poured into vials (10 ml/vial) or bottles (30 ml/bottle), allowed to cool, and stored at 4° C. Flies were maintained at 25° C. and 60% humidity under a 12-h light/dark cycle except where stated otherwise.

Experimental media were identical to standard yeast medium except for the type of yeast extract (Baker's yeast #212750 BACTO™ Yeast Extract, B.D. Diagnost Systems, Sparks, Md.) and the yeast content, at 0.5% w/v (dietary restriction), 2.5% (conventional), and 5.0% (ad libitum). S3QELs at different concentrations in the same volume of DMSO were mixed into a cooled 5% yeast extract medium to give final concentrations of 0.08, 0.8, 8.0, and 80 μM, using DMSO as the vehicle control.

For crosses, 15 virgin females were crossed with 5 male flies in a stock bottle for 5 d, then removed. Larvae were left to develop until eclosion, then flies were transferred to a fresh bottle and aged for 5 d to allow the intestine to mature. Female flies were then sorted under CO₂ anesthesia into experimental vials containing 20-25 flies. For Sod1 and Sod2 knockdown using NP1-GAL4; tub-GAL80ts, flies were crossed and maintained in an incubator at 18° C. Once enclosed, flies were aged for 5 d, sorted onto experimental diets, then maintained in an incubator at 29° C. to induce knockdown.

Lifespan Analysis:

Flies were crossed on standard yeast medium. After eclosion, progeny were transferred to a fresh bottle containing the same medium and aged for 5 d. Mated females were then transferred to experimental diets. For lifespan analysis, 8 vials containing 25 flies were sorted giving a total of 200 flies per condition. Flies were transferred to a fresh vial every other day at which time the numbers of live and dead flies were recorded on an Excel spreadsheet. Lifespan was analyzed in Prism 7 using a Kaplan-Meir analysis to determine median lifespan and a log-rank test to determine statistical significance.

Fly Intestinal Permeability:

Flies that were assayed for lifespan were simultaneously assayed for intestinal permeability. Every 5-10 days flies were transferred to their equivalent experimental diet but containing 2.5% w/v blue food coloring (FD&C #1). After 24 h intestinal permeability was scored on a yes/no basis by the appearance of blue dye in the hemolymph, which made their entire body appear blue. Intestinal permeability is presented as % blue flies within each experimental group.

Acridine Orange/Ethidium Bromide Staining for Intestinal Apoptosis:

12 fly intestines per condition and experimental replicate were dissected into 1× phosphate buffered saline (PBS). Intestines were stained with 5 μg/ml of a 1:1 mix by weight of Acridine Orange/Ethidium Bromide for 5 min, washed 3 times for 10 min with 1×PBS, fixed with 4% v/v paraformaldehyde (PFA) in 1×PBS for 45 min, washed 3 times for 10 min with 1×PBS, then incubated with DAPI for 15 min, and mounted in mowiol mounting medium on a microscope slide. Apoptotic cells were counted from the hindgut-posterior midgut intersection up to the proventriculus using an Olympus BX51 fluorescence microscope.

Phosphohistone H3 (PH3) Staining for Proliferating Cells:

12 fly intestines per condition and experimental replicate were dissected into 1×PBS then fixed with 4% v/v paraformaldehyde in 1×PBS for 45 min, washed for 1 h with wash buffer (1×PBS, 0.5% w/v fatty acid-free bovine serum albumin, and 0.1% v/v Triton X-100), then incubated with rabbit anti-PH3 primary antibody (1:1000, Millipore) in wash buffer overnight at 4° C. The primary antibody was removed and the intestine was washed with wash buffer for 1 h at 4° C., incubated with anti-rabbit Alexa fluor 555 secondary antibody for 4 h at room temperature, washed with wash buffer, stained with DAPI for 15 min, then mounted in mowiol mounting medium on a microscope slide. PH3-positive cells were counted from the hindgut-posterior midgut intersection up to the proventriculus using an Olympus BX51 fluorescence microscope.

Fly Food Consumption:

Food consumption was determined as previously described (Deshpande et al. (2014) Nat. Methods 11: 535-540). Flies maintained on experimental diets for 10 d were transferred to vials containing their equivalent experimental diet but with added 1% w/v blue food coloring (FD&C #1) 1 h after the lights came on in the morning. After 15 min, flies were snap-frozen in liquid nitrogen. Five flies were then homogenized in 100 μL□1×PBS containing 1% v/v Triton X-100. The amount of blue food coloring present in the homogenate was determined by measuring the absorbance at 630 nM.

Mice:

Six-week-old C57BL/6 mice were purchased from Jackson Laboratories and acclimated in-house for two weeks before the study. At 8 weeks of age, mice were randomized into groups based on weight and fed either a control semi-purified diet D12450J containing 10% fat kcal, a 60% fat kcal diet D12492, or a custom-mixed 60% fat kcal diet D12492 containing 200 mg/kg of S3QELs. Diets were purchased from Research Diets and were gamma-irradiated. Mice were maintained on the experimental diets for 16 weeks in total. Food consumption was measured daily in a semiquantitative fashion. The food was weighed before placing in the cage, and then weighed again 24 h later. The total food weight difference within 24 h was divided by the number of mice in the cage to determine average food consumption per mouse. All experiments involving the use of mice were reviewed and approved by the Institutional Animal Care and Use Committee (IACUC) of the Buck Institute.

FITC-Dextran:

Mice were fasted for 4 h prior to assay. Fluorescein isothiocyanate-dextran (FITC-Dextran) molecular weight 4000 (Sigma Aldrich) was administered by oral gavage using a needle attached to a 1 ml syringe at 600 mg/kg body weight. 4 h post FITC-dextran gavaging ˜100 μL of blood was collected via tail bleeding into an EDTA-coated Eppendorf tube and placed on ice. Blood samples were centrifuged for 10 min at 5000 g to isolate blood plasma. Blood plasma samples were diluted appropriately using 1×PBS and FITC-dextran fluorescence was measured at Ex 485 nm/Em 535 nm. FITC-dextran amount was determined using standard curves generated from a known amount of FITC-dextran titrated in plasma from control or high fat-fed mice.

Fecal Albumin:

Feces were collected from individual mice by hand restraining and allowing the mouse to excrete directly into a 1-mL Eppendorf tube. Samples were kept on ice. At assay, fecal samples were diluted to 10 and 100 mg/mL using 1×PBS then centrifuged at 10,000 g for 3 min to pellet fecal debris. 100 μL of the supernatant was assayed to determine albumin concentration using a mouse albumin ELISA (Bethyl Laboratories) according to the manufacturer's guidelines.

Glucose Tolerance:

Mice were fasted for 6 h then a baseline blood glucose concentration was determined before intraperitoneal injection of glucose at 2 mg/kg body weight using an insulin syringe. Blood glucose was measured 15, 30, 45, 60, 90, and 120 min after glucose injection using an Accucheck Aviva glucometer.

Body Fat:

Body composition was determined using an EchoMRI.

Total RNA and cDNA Preparation:

15 fly intestines from each experimental group were dissected (Malpighian tubules removed) and placed into 200 μL of RNA lysis buffer. Fly intestines were manually homogenized using a Kontes microtube pellet pestle rod.

Approximately 50-100 mg of frozen mouse colon or small distal intestine from 10 mice from each experimental group was individually added to 1 mL of TRIzol (Invitrogen) with a stainless steel bead. Intestinal samples were disrupted using a Qiagen TissueLyser Bead Homogenizer for 8 min at 20 Hz. 0.2 mL of chloroform was added, vortexed, left to incubate for 2-3 min, then centrifuged at 12,000 g for 15 min.

RNA from the fly intestine lysates, and the aqueous phase from the mouse intestinal Trizol lysates, were further processed to isolate RNA using a Quick-RNA™ MiniPrep Kit (Zymo Research) according to the manufacturer's guidelines. RNA from the Zymo-spin IIICG column was eluted using 30 μL of DNAse/RNAse-free water by centrifugation for 1 min at 12,000 g. The quantity and quality of fly and mouse RNA samples were determined using a NanoDrop 1000 spectrophotometer (Thermo Scientific). 1 μg of total RNA from either fly or mouse intestine was used in a total reaction volume of L containing 4 μL of iScript Reverse Transcription Supermix (Bio-Rad) to synthesize cDNA according to the manufacturer's guidelines. cDNA was stored at −20° C. before carrying out quantitative RTPCR.

Quantitative Real-Time PCR (qPCR):

A 10 μL reaction volume containing 200 ng of cDNA, 400 nM of forward and reverse primer and 5 μL of SensiFAST SYBR No-ROX Kit (BIOLINE) was used in a qPCR reaction performed using a Light Cycler 480 Real-Time PCR System (Roche Applied Science). For the fly intestinal samples, fold changes in gene expression were determined using the 2\hat{ }(−ΔΔCt) method. Gene expression was normalized to RpL32. For the mouse intestinal samples, the mean ΔCt value for the control group was determined, and the control mouse ΔCt value closest to the mean of the entire group was used as the calibrator to determine gene expression fold change using the 2\hat{ }(−ΔΔCt) method. Gene expression was normalized to β-actin.

Statistical Analysis:

Data are means±SEM. Differences between groups were analyzed by either one-way ANOVA with Tukey's test, or two-way ANOVA with Dunnett's multiple comparison test as appropriate. Pair-wise analyses of median lifespan were conducted by paired two-tailed t test. Lifespans were analyzed by Log Rank (Mantel-Cox) test. Statistics were analyzed and graphs were generated using Microsoft Excel and GraphPad Prism.

TABLE 3
Drosophila primers.

| | | SEQ |
|---|---|---|
| Primer | | ID |
| name | Primer Sequence | NO |
| RpL32 5′ | CAACAGAGTCGGTCGCCGCTTC | 1 |
| RpL32 3′ | CAGCTCGCGCACGTTGTGCACC | 2 |
| Dpt 5′ | CACGAGATTGGACTGAATGG | 3 |
| Dpt 3′ | TTTCCAGCTCGGTTCTGAGT | 4 |
| Drs 5′ | GAGGAGGGACGCTCCAGT | 5 |
| Drs 3′ | TTAGCATCCTTCGCACCAG | 6 |
| Def 5′ | TTTTGCTCTGCTTGCTTGC | 7 |
| Def 3′ | ACATGATCCTCTGGAATTGGA | 8 |
| upd3 5′ | ACCTACAGAAGCGTTCCAG | 9 |
| upd3 3′ | GGTTCTGTAGATTCTGCAGG | 10 |
| Tsp2A 5′ | GTAATGATGGCCGTCAGCTT | 11 |
| Tsp2A 3′ | TGGTCAGCACGTAGTTCGAG | 12 |
| Ssk 5′ | TTCGGCACAACCAAACATAAG | 13 |
| Ssk 3′ | GTGGTTCGCACAGCTCTCT | 14 |
| mesh 5′ | ACCAAAAGCGGTTGACATTC | 15 |
| mesh 3′ | CAGGGAATTCAGCTGGGATA | 16 |
| Bbg 5′ | GTGGTGTCAACGATGTCCT | 17 |
| Bbg 3′ | ACCGCAGTACGGTGAATAGG | 18 |
| Cora 5′ | GGGTCCACCTATCGCTACAA | 19 |
| Cora 3′ | CAGAGCTGAAAGGATCAGGT | 20 |
| Pyd 5′ | CACCACCTCGTCGTATTCCT | 21 |
| Pyd 3′ | GACTGCCGTACAACCAGGAT | 22 |
| Crb 5′ | CAGGAGCAGCAATCTGACGA | 23 |
| Crb 3′ | GGTCACTCGTCCTCCGTTTA | 24 |
| Baz 5′ | ATCATGCTGGTCATGGTCAA | 25 |
| Baz 3′ | GTCCGGCAGAGCAGTTAGT | 26 |

TABLE 4
Mouse PCR primers.

| | | SEQ |
|---|---|---|
| | | ID |
| Primer name | Primer Sequence | NO |
| □-Actin 5′ | AGCACTGTGTTGGCATAGAG | 27 |
| | GTC | |
| □-Actin 3′ | CTTCTTGGGTATGGAATCCT | 28 |
| | GTG | |
| ZO-1 5′ | CCACCTCTGTCCAGCTCTTC | 29 |
| ZO-1 3′ | CACCGGAGTGATGGTTTTCT | 30 |
| Occludin 5′ | TTGAAAGTCCACCTCCTTACA | 31 |
| | GA | |
| Occludin 3′ | CCGGATAAAAAGAGTACGCTGG | 32 |
| JAM-A 5′ | TCTCTTCACGTCTATGATCCT | 33 |
| | GG | |
| JAM-A 3′ | TTTGATGGACTCGTTCTCGGG | 34 |
| Claudin 5 5′ | GCAAGGTGTATGAATCTGTGCT | 35 |
| Claudin 5 3′ | GTCAAGGTAACAAAGAGTGCCA | 36 |
| Muc2 5′ | ATGCCCACCTCCTCAAAGAC | 37 |
| Muc2 3′ | GTAGTTTCCGTTGGAACAGTGA | 38 |
| Muc4 5′ | GCTCAAGTTGACAAGGAGC | 39 |
| | AGAGC | |
| Muc4 3′ | GGAGGACAAAAGAAGGCGTGG | 40 |
| | CC | |
| Muc13 5′ | GCCAGTCCTCCCACCACGGTA | 41 |
| Muc13 3′ | CTGGGACCTGTGCTTCCACCG | 42 |
| Klf4 5′ | AGCCACCCACACTTGTGAC | 43 |
| | TATG | |
| Klf4 3′ | CAGTGGTAAGGTTTCTCGC | 44 |
| | CTGTG | |
| sXbp1 5′ | GAGTCCGCAGCAGGTGC | 45 |
| sXbp1 3′ | CAAAAGGATATCAGACTCAG | 46 |
| | AATCTGAA | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for the treatment or prophylaxis of an age-related and/or pathology-associated increase in intestinal barrier permeability, said method comprising:
administering to a mammal in need thereof an effective amount of one or more agent(s) that inhibit superoxide production from the outer ubiquinone-binding site of complex III of the mitochondrial electron transport chain (site III_{Qo}).

2. The method of claim 1, wherein said agent comprises an agent that partially or fully suppresses superoxide generation at complex III of the mitochondrial electron transport chain without inhibiting oxidative phosphorylation.

3. The method of claim 2, wherein said agent comprises a small-molecule suppressor of site III_{Qo} electron leak (a S3QEL).

4. The method according to any one of claims 1-3, wherein said agent comprises a sulfanyloxoquinazoline structural group S3QEL.

5. The method of claim 4, wherein said agent comprises a S3QEL selected from the group consisting of S3QEL-1, S3QEL-1.1, S3QEL-1.2, and S3QEL-1.3.

6. The method according to any one of claims 1-5, wherein said agent comprises a pyrazolopyrimidine structural group S3QEL.

7. The method of claim 6, wherein said agent comprises a S3QEL selected from the group consisting of S3QEL-2, S3QEL-2.1, S3QEL-2.2, S3QEL-2.3, S3QEL-2.4, S3QEL-2.5, S3QEL-2.6, S3QEL-2.7, S3QEL-2.8.

8. The method according to any one of claims 1-7, wherein said agent comprises a S3QEL selected from the group consisting of S3QEL-4, S3QEL-5, S3QEL-6, and S3QEL-7.

9. The method according to any one of claims 1-3, wherein said agent comprises S3QEL-1, S3QEL-1.1, S3QEL-1.2, and S3QEL-1.3.

10. The method of claim 9, wherein said agent comprises S3QEL-2.

11. The method according to any one of claims 9-10, wherein said agent comprises S3QEL-2.1.

12. The method according to any one of claims 9-11, wherein said agent comprises S3QEL-2.2.

13. The method according to any one of claims 9-12, wherein said agent comprises, S3QEL-2.3.

14. The method according to any one of claims 9-13, wherein said agent comprises S3QEL-2.4.

15. The method according to any one of claims 9-14, wherein said agent comprises S3QEL-2.5.

16. The method according to any one of claims 9-15, wherein said agent comprises S3QEL-2.6.

17. The method according to any one of claims 9-16, wherein said agent comprises S3QEL-2.7.

18. The method according to any one of claims 9-17, wherein said agent comprises S3QEL-2.8.

19. The method according to any one of claims 9-18, wherein said agent comprises S3QEL-4.

20. The method according to any one of claims 9-19, wherein said agent comprises S3QEL-5.

21. The method according to any one of claims 9-20, wherein said agent comprises S3QEL-6.

22. The method according to any one of claims 9-21, wherein said agent comprises S3QEL-7.

23. The method according to any one of claims 1-22, wherein said agent(s) are provided in a delivery vehicle compatible with a hydrophobic compound, a pharmaceutically acceptable solvate, a pharmaceutically acceptable ester or ether, or a pharmaceutically acceptable clathrate.

24. The method of claim 23, wherein said agent(s) are provided as a lipid or liposome formulation.

25. The method according to any one of claims 1-24, wherein said method provides treatment or prophylaxis for diet-induced intestinal permeability.

26. The method of claim 25, wherein said method provides treatment for diet-induced intestinal permeability in mammal that is obese or clinically obese.

27. The method of claim 25, wherein said method provides treatment for diet-induced intestinal permeability in a mammal that is diabetic.

28. The method of claim 25, wherein said method provides prophylaxis for diet-induced intestinal permeability in a mammal that is obese or clinically obese.

29. The method of claim 25, wherein said method provides prophylaxis for diet-induced intestinal permeability in mammal that is diabetic.

30. The method according to any one of claims 1-24, wherein said method provides treatment for age-related increases in intestinal permeability.

31. The method according to any one of claims 1-24, wherein said method provides treatment or prophylaxis for a pathology-associated increase in intestinal barrier permeability.

32. The method of claim 31, wherein said pathology comprises a pathology directly associated with the gut.

33. The method of claim 32, wherein said pathology comprise a pathology selected from the group consisting of a gastric ulcer, infectious diarrhea, irritable bowel syndrome (IBS), inflammatory bowel disease (IBD), celiac disease, cancer associated with digestive tract (esophagus, stomach, colorectal), colitis, Crohn's disease, mitochondrial neurogastrointestinal encephalopathy (MNGIE), and hyperintestinal permeability.

34. The method of claim 33, wherein said mammal is a mammal identified as having a gastric ulcer.

35. The method of claim 33, wherein said mammal is a mammal identified as having infectious diarrhea.

36. The method of claim 33, wherein said mammal is a mammal identified as having irritable bowel syndrome (IBS).

37. The method of claim 33, wherein said mammal is a mammal identified as having inflammatory bowel disease (IBD).

38. The method of claim 33, wherein said mammal is a mammal identified as having celiac disease.

39. The method of claim 33, wherein said mammal is a mammal identified as having cancer associated with digestive tract (e.g., esophagus, stomach, colorectal).

40. The method of claim 33, wherein said mammal is a mammal identified as having colitis.

41. The method of claim 33, wherein said mammal is a mammal identified as having Crohn's disease.

42. The method of claim 33, wherein said mammal is a mammal identified as having mitochondrial neurogastrointestinal encephalopathy (MNGIE).

43. The method of claim 33, wherein said mammal is a mammal identified as having hyperintestinal permeability.

44. The method of claim 31, wherein said pathology comprises a pathology indirectly associated with the gut.

45. The method of claim 44, wherein said pathology comprise a pathology selected from the group consisting of a respiratory infection, acute inflammation (sepsis, SIRS, MOF), chronic inflammation, and an obesity-associated metabolic disease (e.g., NASH, diabetes type I and II, CVD).

46. The method of claim 45, wherein said mammal is a mammal identified as having a respiratory infection.

47. The method of claim 45, wherein said mammal is a mammal identified as having an acute inflammation (sepsis, SIRS, MOF).

48. The method of claim 45, wherein said mammal is a mammal identified as having chronic inflammation.

49. The method of claim 45, wherein said mammal is a mammal identified as having an obesity-associated metabolic disease.

50. The method of claim 49, wherein said obesity-associated metabolic disease comprises one or more pathologies selected form the group consisting of (NASH, type I diabetes, type II diabetes, and CVD.

51. The method according to any one of claims 33-50, wherein said method ameliorates one or more symptoms of said pathology.

52. The method according to any one of claims 1-51, wherein said method is a therapeutic method to reduce intestinal permeability, or to slow or stop an increase in intestinal permeability.

53. The method according to any one of claims 1-51, wherein said method is a prophylactic method effective to delay or to prevent an onset of increase in intestinal permeability.

54. The method according to any one of claims 1-53, wherein said agent(s) do not decrease food consumption by said mammal.

55. The method according to any one of claims 1-54, wherein said agent(s) decrease the number of apoptotic cells in the intestine.

56. The method according to any one of claims 1-55, wherein said agent(s) decrease intestinal damage.

57. The method according to any one of claims 1-56, wherein said agent(s) decrease intestinal permeability.

58. The method of claim 57, wherein said decrease in intestinal permeability comprises a decrease in intestinal permeability as measured by presence of non-absorbable markers in serum after ingestion.

59. The method of claim 58, wherein said nonabsorbable markers are selected form the group consisting of nonabsorbable sugars, dextran, and PEGs.

60. The method of claim 59, wherein said nonabsorbable markers are selected from the group consisting of lactose, mannitol, L-rhamnose, sucralose, and erythritol.

61. The method of claim 59, wherein said nonabsorbable markers comprise FITC-dextran.

62. The method according to any one of claims 57-60, wherein said decrease in intestinal permeability comprises a decrease in intestinal permeability as measured by the presence or level of permeability biomarkers.

63. The method of claim 62, wherein said biomarkers comprise a biomarker selected from the group consisting of plasma zonulin, calprotectin, and alpha-1 antitrypsin (A1AT).

64. The method according to any one of claims 57-63, wherein said decrease in intestinal permeability comprises a decrease in intestinal permeability as measured by plasma lipopolysaccharide load.

65. The method according to any one of claims 57-64, wherein said decrease in intestinal permeability comprises a decrease in intestinal permeability as measured by circulating endotoxin core antibodies (EndoCAb) and/or plasma D-lactate level, and/or fecal butyrate concentration.

66. The method according to any one of claims 57-65, wherein said decrease in intestinal permeability comprises a decrease in intestinal permeability as measured by the presence or level of inflammatory cytokines.

67. The method of claim 66, wherein said inflammatory cytokines comprise a cytokine selected from the group consisting of TNFα, INFγ, IL-1β, and IL-13.

68. The method according to any one of claims 57-67, wherein said agent(s) decreases the expression of antimicrobial protein genes.

69. The method of claim 68, wherein said antimicrobial protein genes include one or more genes selected from the group consisting of Dpt, Drs and Def, and upd3.

70. The method according to any one of claims 1-69, wherein said agent(s) act in intestinal enterocytes.

71. The method of claim 70, wherein said agent(s) act specifically in intestinal enterocytes.

72. The method according to any one of claims 1-71, wherein administering said agent(s) decreases weight gain.

73. The method according to any one of claims 1-72, wherein said agent(s) are administered via a route selected from the group consisting of intraperitoneal administration, oral administration, inhalation administration, transdermal administration, subdermal depot administration, and rectal administration.

74. The method of claim 73, wherein said agent(s) are administered orally.

75. The method according to any one of claims 73-74, wherein said agent(s) are administered as a unit dosage formulation.

76. The method according to any one of claims 1-75, wherein said mammal is a human.

77. The method according to any one of claims 1-75, wherein said mammal is a non-human mammal.

78. A kit for the treatment or prophylaxis of an age-related and/or pathology-associated increase in intestinal barrier permeability, said kit comprising:
one or more agent(s) that inhibit superoxide production from the outer ubiquinone-binding site of complex III of the mitochondrial electron transport chain (site III_{Qo}); and
instructional materials describing the use of the active agent(s) in a method for the treatment or prophylaxis of an age-related and/or pathology-associated increase in intestinal barrier permeability.

79. The kit of claim 78, wherein said instructional materials teach the use of said agent(s) in a method according to any one of claims 1-77.

80. The kit according to any one of claims 78-79, wherein said agent(s) comprise one or more S3QELs selected from the group consisting of S3QEL-1, S3QEL-1.1, S3QEL-1.2, and S3QEL-1.3, S3QEL-2, S3QEL-2.1, S3QEL-2.2, S3QEL-2.3, S3QEL-2.4, S3QEL-2.5, S3QEL-2.6, S3QEL-2.7, S3QEL-2.8, S3QEL-4, S3QEL-5, S3QEL-6, and S3QEL-7.


SEQUENCE LISTING (46 sequences)

SEQ ID NO: 1 - DNA, 22 bp, Artificial
  PCR primer
caacagagtcggtcgccgcttc

SEQ ID NO: 2 - DNA, 22 bp, Artificial
  PCR primer
cagctcgcgcacgttgtgcacc

SEQ ID NO: 3 - DNA, 20 bp, Artificial
  PCR primer
cacgagattggactgaatgg

SEQ ID NO: 4 - DNA, 20 bp, Artificial
  PCR primer
tttccagctcggttctgagt

SEQ ID NO: 5 - DNA, 18 bp, Artificial
  PCR primer
gaggagggacgctccagt

SEQ ID NO: 6 - DNA, 19 bp, Artificial
  PCR primer
ttagcatccttcgcaccag

SEQ ID NO: 7 - DNA, 19 bp, Artificial
  PCR primer
ttttgctctgcttgcttgc

SEQ ID NO: 8 - DNA, 21 bp, Artificial
  PCR primer
acatgatcctctggaattgga

SEQ ID NO: 9 - DNA, 19 bp, Artificial
  PCR primer
acctacagaagcgttccag

SEQ ID NO: 10 - DNA, 20 bp, Artificial
  PCR primer
ggttctgtagattctgcagg

SEQ ID NO: 11 - DNA, 20 bp, Artificial
  PCR primer
gtaatgatggccgtcagctt

SEQ ID NO: 12 - DNA, 20 bp, Artificial
  PCR primer
tggtcagcacgtagttcgag

SEQ ID NO: 13 - DNA, 21 bp, Artificial
  PCR primer
ttcggcacaaccaaacataag

SEQ ID NO: 14 - DNA, 19 bp, Artificial
  PCR primer
gtggttcgcacagctctct

SEQ ID NO: 15 - DNA, 20 bp, Artificial
  PCR primer
accaaaagcggttgacattc

SEQ ID NO: 16 - DNA, 20 bp, Artificial
  PCR primer
cagggaattcagctgggata

SEQ ID NO: 17 - DNA, 19 bp, Artificial
  PCR primer
gtggtgtcaacgatgtcct

SEQ ID NO: 18 - DNA, 20 bp, Artificial
  PCR primer
accgcagtacggtgaatagg

SEQ ID NO: 19 - DNA, 20 bp, Artificial
  PCR primer
gggtccacctatcgctacaa

SEQ ID NO: 20 - DNA, 20 bp, Artificial
  PCR primer
cagagctgaaaggatcaggt

SEQ ID NO: 21 - DNA, 20 bp, Artificial
  PCR primer
caccacctcgtcgtattcct

SEQ ID NO: 22 - DNA, 20 bp, Artificial
  PCR primer
gactgccgtacaaccaggat

SEQ ID NO: 23 - DNA, 20 bp, Artificial
  PCR primer
caggagcagcaatctgacga

SEQ ID NO: 24 - DNA, 20 bp, Artificial
  PCR primer
ggtcactcgtcctccgttta

SEQ ID NO: 25 - DNA, 20 bp, Artificial
  PCR primer
atcatgctggtcatggtcaa

SEQ ID NO: 26 - DNA, 19 bp, Artificial
  PCR primer
gtccggcagagcagttagt

SEQ ID NO: 27 - DNA, 23 bp, Artificial
  PCR primer
agcactgtgttggcatagaggtc

SEQ ID NO: 28 - DNA, 23 bp, Artificial
  PCR primer
cttcttgggtatggaatcctgtg

SEQ ID NO: 29 - DNA, 20 bp, Artificial
  PCR primer
ccacctctgtccagctcttc

SEQ ID NO: 30 - DNA, 20 bp, Artificial
  PCR primer
caccggagtgatggttttct

SEQ ID NO: 31 - DNA, 23 bp, Artificial
  PCR primer
ttgaaagtccacctccttacaga

SEQ ID NO: 32 - DNA, 22 bp, Artificial
  PCR primer
ccggataaaaagagtacgctgg

SEQ ID NO: 33 - DNA, 23 bp, Artificial
  PCR primer
tctcttcacgtctatgatcctgg

SEQ ID NO: 34 - DNA, 21 bp, Artificial
  PCR primer
tttgatggactcgttctcggg

SEQ ID NO: 35 - DNA, 22 bp, Artificial
  PCR primer
gcaaggtgtatgaatctgtgct

SEQ ID NO: 36 - DNA, 22 bp, Artificial
  PCR primer
gtcaaggtaacaaagagtgcca

SEQ ID NO: 37 - DNA, 20 bp, Artificial
  PCR primer
atgcccacctcctcaaagac

SEQ ID NO: 38 - DNA, 22 bp, Artificial
  PCR primer
gtagtttccgttggaacagtga

SEQ ID NO: 39 - DNA, 24 bp, Artificial
  PCR primer
gctcaagttgacaaggagcagagc

SEQ ID NO: 40 - DNA, 23 bp, Artificial
  PCR primer
ggaggacaaaagaaggcgtggcc

SEQ ID NO: 41 - DNA, 21 bp, Artificial
  PCR primer
gccagtcctcccaccacggta

SEQ ID NO: 42 - DNA, 21 bp, Artificial
  PCR primer
ctgggacctgtgcttccaccg

SEQ ID NO: 43 - DNA, 23 bp, Artificial
  PCR primer
agccacccacacttgtgactatg

SEQ ID NO: 44 - DNA, 24 bp, Artificial
  PCR primer
cagtggtaaggtttctcgcctgtg

SEQ ID NO: 45 - DNA, 17 bp, Artificial
  PCR primer
gagtccgcagcaggtgc

SEQ ID NO: 46 - DNA, 28 bp, Artificial
  PCR primer
caaaaggatatcagactcagaatctgaa